(19) United States
(12) Patent Application Publication
(10) Pub. No.: US 2019/0004070 A9
(43) Pub. Date: Jan. 3, 2019

(54) DUAL ANTI-PLATELET MEDICATION/ASPIRIN RESPONSE AND REACTIVITY TEST USING SYNTHETIC COLLAGEN

(71) Applicant: JNC CORPORATION, Tokyo (JP)

(72) Inventor: William M. TROLIO, Horsham, PA (US)

(73) Assignee: JNC CORPORATION, Tokyo (JP)

(21) Appl. No.: 15/874,780

(22) Filed: Jan. 18, 2018

Related U.S. Application Data
(62) Division of application No. 14/419,880, filed on Feb. 5, 2015, now abandoned.
(60) Provisional application No. 61/681,485, filed on Aug. 9, 2012.
(60) Provisional application No. 61/680,111, filed on Aug. 6, 2012.

(51) Int. Cl. G01N 33/86

(52) U.S. Cl. CPC G01N 33/86; G01N 2333/78; G01N 2800/52

(57) ABSTRACT

The present invention provides tests that measures functional platelet aggregation such as by using Light Transmission Aggregometry Assays (LTAAs) or flow cytometry, using synthetic, self-assembling human type I collagen, methods of predicting and measuring an individual's platelet anti-platelet medication sensitivity and residual platelet activity status when the individual is on a dual anti-platelet therapy of aspirin and anti-platelet medication and kits useful in the assays and methods.

This application is a divisional of U.S. patent application Ser. No. 14/419,880 filed on Feb. 5, 2015 (pending), which is a U.S. National Phase under 35 U.S.C. § 371 of International Application PCT/US2013/053612, filed on Aug. 5, 2013, which claims priority to U.S. Provisional Application 61/680,111, filed on Aug. 6, 2012, and U.S. Provisional Applications 61/681,485 filed on Aug. 9, 2012, which are incorporated herein in their entirety. This application also claims priority to PCT application, PCT/US13/49418, filed on Jul. 5, 2013. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### BACKGROUND OF THE INVENTION

The conventional, primary need for an effective assessment of platelet response and reactivity is in the field of cardiology. The public health incidence and burden of heart attack, stroke and related cardiovascular and thrombotic diseases are well known. The medical community has long recommended the use of aspirin in primary care to reduce cardiovascular, stroke and certain other risks. The use of Aspirin in other area such as DVT prophylaxis, oncology, orthopedics and prevention is driving a renewed interest in and use of Aspirin alone or in combination with other drugs. Additionally compliance testing and personalized medicine initiatives increase the unmet medical need for a test which can provide Aspirin presence and response, presence and efficacy of a second antiplatelet drug and the residual reactivity of a patient's platelet reactivity. Aspirin (salicylate based compounds) ingestion or exposure inhibits the COX 1 pathway and modifies COX 2 enzymatic processes, which then precludes all subsequent events necessary for platelet aggregation. Since the ingestion of or exposure to aspirin can inhibit platelet aggregation, it has been given as a therapy to prevent undesired platelet aggregation, which can be a source of many heart attacks, strokes and other thrombotic events.

Despite the benefits of aspirin therapy in many individuals, aspirin therapy is not effective enough in some individuals as it does not cause the desired inhibition of platelet aggregation or its effect is shorter than the dosing interval (some patients may only get 6 to 12 hours of protection rather than 24 hours resulting in an above baseline risk for the patient in the time between doses). In these individuals the residual platelet reactivity is high and the patient's risk is not mitigated. In other individuals, aspirin therapy can be harmful as it creates an increased risk of unwanted bleeding complications because the aspirin seems to block platelet activity altogether so that the blood does not clot when physiologically necessary. Recently, two elements of Aspirin's pharmacodynamic behavior have entered the clinical mindset: Aspirin must be taken at the same time every day to maintain its antiplatelet effectiveness; and the thrombotic risk from failure to keep this schedule is greater than the patient's baseline risk.

Thus, physicians often prescribe a course of treatment involving both low dose aspirin and an anti-platelet medication to inhibit unwanted platelet aggregation. This is often called a “dual therapy.” Not all patients will respond to the dual therapy or the individual anti-platelet medication in the same way. Selecting and monitoring Aspirin in dual treatment protocols is also necessary because certain antiplatelet drugs like ticagrelor lose their effectiveness if the Aspirin dose is greater than 100 mg. There currently is not an effective way to measure a patient's response to dual anti-platelet medication therapy or to determine the patient's residual platelet reactivity. Thus, there is an unmet need for a reliable tool to assess and manage the anti-platelet medication response and reactivity on platelet aggregation when a patient is on a dual anti-platelet medication therapy, as well as checking patient compliance with the treatment regimen.

Traditionally, a patient's response to aspirin and other anti-platelet medication therapy is assessed by testing platelet activity using a series of platelet aggregation test. The “gold standard” of platelet aggregation tests, light transmission aggregometry (LTA), utilizes collagen from biological sources as the agonist to bring about platelet aggregation, as a measure of the degree or extent of platelet response or inhibition to aggregation. However, there are multiple issues as well as the risk of infectious disease transmission when using biological material. Biologically derived products, whether ‘natural,’ processed, manufactured by fermentation, cell culture or similar processes, or recombinant, all share the following drawbacks: carry a risk of infectious disease transmission; have lot to lot variability (regarding the ratio of active materials, performance, chemical characteristics, solubility, stability, moisture content, and process contaminants); differing bio-profiles depending upon the location the product was made; differences caused by processing; and environment, geographic and dietary differences affecting the source animal or culture. Biological collagens cannot be diluted to enhance sensitivity or to generate dilution profiles, both of which easily performed with synthetic collagen.

It is widely believed that anti-platelet therapy contributes to the reduction of major atherothrombotic complications in cardiovascular, neurovascular and other diseases. In the treatment of percutaneous coronary intervention and acute coronary syndromes, dual anti-platelet therapy when performed at optimal dosing and timing has significantly lowered the risk of thrombotic complications and contributed to positive outcomes. However, an important clinical problem of increased incidence of major adverse clinical events (“MACE”) and confounding differences in patient outcomes relates to the variability in patient response to anti-platelet treatments, especially in a dual antiplatelet therapy (a combination of Aspirin and a second agent such as clopidogrel or ticagrelor). Understanding the mechanisms underlying this phenomenon is important to individualizing and improving patient care, long term (maintenance—sometimes referred to as chronic therapy in the literature) therapy and consistent (positive) outcomes.

However, a clear and reliable predictive model for responsiveness to anti-platelet therapy is currently not available. Attempts have been made to characterize the efficacy of anti-platelet therapy using platelet function testing but based on current information, and dependence on biological agonists, its routine use is not recommended particularly as costs, complexities and cost effectiveness have not been established, and lack of correlation, standardization and agreement between laboratory methods, and their respective results, is well documented in the literature. Tobias Geislera et al., Circulation 2010; 122:1049-1052. In addition, the inhibitory effects of aspirin on platelets decreases over time in patients on long term or chronic therapy. Violi, F. et al., J Am Cardio, Vol. 43, No. 6, 2004. Further, as evidenced in AstraZeneca's complete prescribing information on ticagrelor (trade name Brilinta® in the US, Brilique® and Possia® in Europe), Aspirin doses greater than 100 mg reduce the ability of ticagrelor to inhibit platelet aggregation. This conflicts with current clinical care guidelines in which increasing doses of Aspirin to ‘overcome’ Aspirin resistance is recommended.

Thus, there remains a need for a quantitative, functional platelet activity test that does not use an animal derived collagen as the agonist. There remains a need for a test that is able to measure the platelet response to an anti-platelet medication when the patient is on a dual therapy of aspirin and an anti-platelet medication, as well as a test to monitor patient compliance with the dual therapy regimen. Dual antiplatelet therapy is the global practice standard. There is also a need for a test that can measure residual platelet activity (that is the platelet activity that remains even while the patient is on a dual anti-platelet medication therapy) rather than platelet inhibition, which is the result for currently available tests. Platelet inhibition does not equal residual platelet reactivity. Residual platelet reactivity is what determines patient outcome and thus is the better measurement. The present invention meets these needs.

### SUMMARY OF THE INVENTION

The present invention provide tests for determining a donor's platelet combined sensitivity status when the individual is on a dual therapy of aspirin and an anti-platelet medication comprising the use of synthetic collagen. Exemplary tests include the use of platelet aggregation studies using for example, light transmission aggregation assays (LTAAs) and flow Cytometry, and to a lesser degree, methods based on measuring aggregation by impedance or whole blood aggregometry. The tests of the present invention use synthetic collagen as the agonist.

Tests of the invention provide assays that “discount” or “ignore” the effect of the aspirin on platelet aggregation, but still measure the effect of the anti-platelet medication on the platelet aggregation. In these embodiments the final in-test concentration of synthetic collagen that is used tests the ability of the platelets to aggregate in the presence of an agonist (synthetic collagen) and is at a concentration where the test ignores the effect of aspirin on platelet aggregation but still measures the effect of the anti-platelet medication on the platelet aggregation. Preferably, in these embodiments the final in-test concentration of synthetic collagen used ranges from about 50 ng/mL to about 500 ng/mL; or is >40 ng/mL; or is >50 ng/mL; or ranges from about 40 to about 500 ng/mL; or ranges from about 40 to about 400 ng/mL; or ranges from about 40 to 300 ng/mL; or ranges from about 40 to about 200 ng/mL; or ranges from about 40 to about 100 ng/mL; or ranges from about 40 to about 90 ng/mL; or ranges from about 40 to about 80 ng/mL; or ranges from about 40 to about 70 ng/mL; or ranges from about 40 to about 60 ng/mL; or ranges from about 50 to about 400 ng/mL; or ranges from about 50 to about 300 ng/mL; or ranges from about 50 to about 200 ng/mL; or ranges from about 50 to about 100 ng/mL.

Further, the invention provides tests that “discount” or “ignore” the effect of the anti-platelet medication, but still measure the effect of the aspirin on platelet aggregation. In these embodiments the final in-test concentration of synthetic collagen that is used tests the ability of the platelets to aggregate in the presence of an agonist (synthetic collagen) and is at a concentration where the test ignores the effect of the anti-platelet medication on platelet aggregation but still measures the effect of the aspirin on the platelet aggregation. Preferably, in these embodiments the final in-test concentration of synthetic collagen used ranges from about 0.01 ng/mL to about 1.0 ng/mL; or ranges from about 0.1 ng/mL to about 0.5 ng/mL; or ranges from about 0.1 ng/mL to about 1.0 ng/mL; or ranges from about 0.1 ng/mL to about 1.5 ng/mL; or is from about 0.5 ng/mL or less; or ranges from about 0.5 ng/mL to about 2.0 ng/mL; or is less than 2.0 ng/mL; or is less than 10 ng/mL; or is less than 5 ng/mL.

The methods of the present invention are able to test the ability of the individual's platelets to aggregate after the individual has ingested an anti-platelet medication and aspirin. In other words, this tests the residual platelet activity—how reactive are the platelets (likely to aggregate) after the patient has ingested the dual therapy of an anti-platelet medication and aspirin. In these embodiments, the concentration of synthetic collagen is such that the effect of both the anti-platelet medication and the aspirin on platelet aggregation is taken into account and the activity of the platelets is the activity that remains after the medications have had their effect.

Preferably in these embodiments the final in-test concentration of synthetic collagen used preferably ranges from about 25 to 35 ng/mL; or is about 2.0 ng/mL; or ranges from 2.0 to 12.5 ng/mL; or ranges from about 2.0 to about 25 ng/mL; or ranges from about 2.0 to about 35 ng/mL; or ranges from about 2.0 to about 39 ng/mL; or is about 12.5 ng/mL; or ranges from about 12.5 to about 25 ng/mL; or ranges from about 12.5 to about 35 ng/mL; or ranges from about 12.5 to about 39.0 ng/mL; or ranges from about 25 to about 39 ng/mL.

The literature shows that 30-70% of patients do not comply with taking their medication or taking it as directed, and there is no current compliance test. There is developing thought that ‘resistance’ may be due, at least in part, to non-compliance.

The present invention also provides a method of testing patient compliance, with the dual therapy regimen. The DAPT results will show noncompliance for Aspirin therapy as well as deviation from the scheduled dosing window. The present invention also provides a method of testing patient compliance for the aspirin therapy aspect while on a dual therapy, or testing patient compliance for the anti-platelet medication aspect while on a dual therapy.

The present invention also provides methods of predicting the effectiveness of aspirin therapy, of an anti-platelet medication therapy, or a dual therapy.

In certain embodiments, the synthetic collagen is a synthetic collagen that has the ability to self-assemble into a triple helix to form fibrils and which mimics human type I collagen. In certain embodiments the synthetic collagen comprises a polypeptide having a peptide fragment represented by the formula (I)

-(Pro-X-Gly)ₙ (I)

wherein X represents Hyp; and n represents an integer of from 20 to 5,000; and

wherein the polypeptide has a molecular weight at a range of from 10,000 to 500,000. In certain embodiments, n=20-250.

The present invention also provides kits for testing platelet aggregation in a light transmission assay, comprising a vial of synthetic collagen; and instructions for use of the synthetic collagen in the dual anti-platelet therapy test (DAPT) of the present invention.

In certain embodiments (including the methods described herein and the kits), the synthetic collagen is supplied and/or stored in a polypropylene homomer container. In certain embodiments, the cap is the same material as the vial/tube. In certain embodiments, the container has an additional internal seal or a cap having a secondary seal molded therein. In certain embodiments, the container contains all of the above described characteristics

### BRIEF DESCRIPTION OF THE FIGURES

FIG. 1 provides graphs showing the use of a high concentration of synthetic collagen, which shows at high concentrations, the synthetic collagen has no sensitivity to aspirin.

FIG. 2 provides graphs showing the use of a low concentration of synthetic collagen, which shows that at low concentrations synthetic collagen is sensitive to aspirin. It is this unique property of the synthetic collagen that is the basis for responsiveness and residual reactivity determinations as well as the concurrent assessment of multiple classes of anti-platelet medications.

FIG. 3 shows the results of LTAs run using synthetic collagen. This figure shows that synthetic collagen provides the ability to measure the effect of other anti-platelet medications in patients on a dual anti-platelet therapy (when one medication is aspirin).

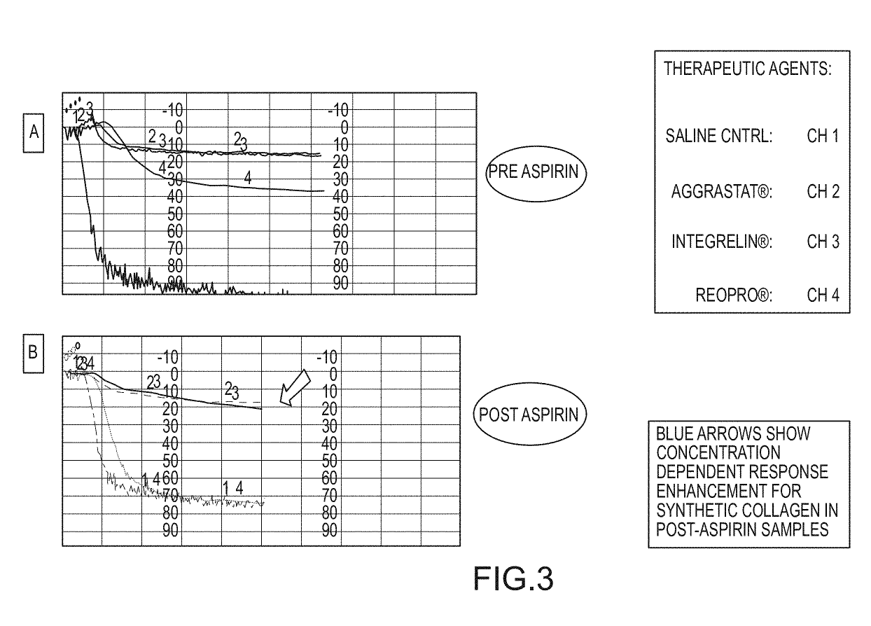

FIG. 4 provides a graph of test (with the readout as area under the curve (“AUC”)) run on an individual known to be aspirin resistant before and after aspirin ingestion. The tests were run over various dilutions of synthetic collagen. A “bounce back” is seen between 1.0 ng/mL and 0.1 ng/mL. This bounce back is where the line showing the AUC should be decreasing down to correspond with the decreasing concentration of synthetic collagen, but instead bounces up and then bounces down to where it should be (decreasing).

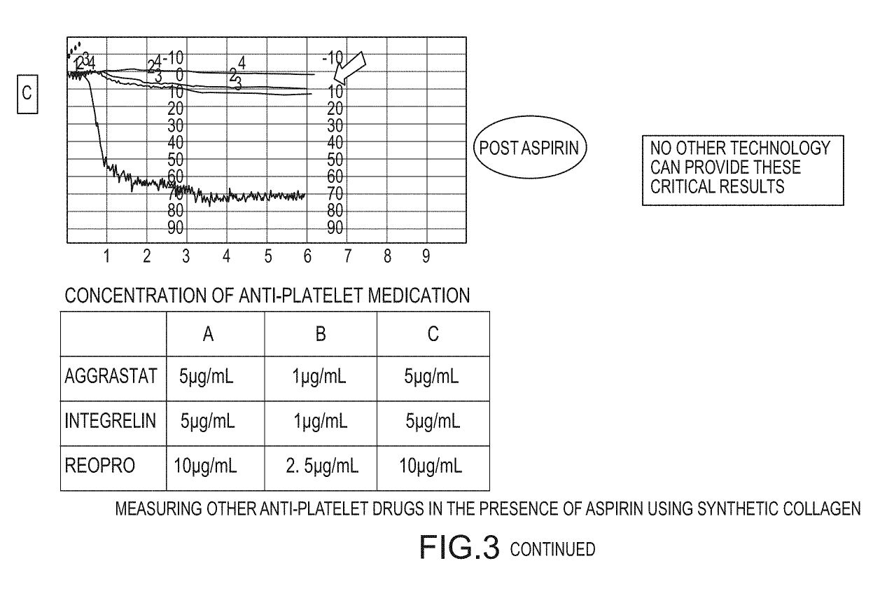

FIG. 5 provides a graph of test (with the readout as area under the curve (“AUC”)) run on an individual known to be a normal or average aspirin responder before and after aspirin ingestion. The tests were run over various dilutions of synthetic collagen. As expected the AUC decreases as the concentration of synthetic collagen decreases. There is no pronounced “bounce back” as seen in the aspirin resistant individual.

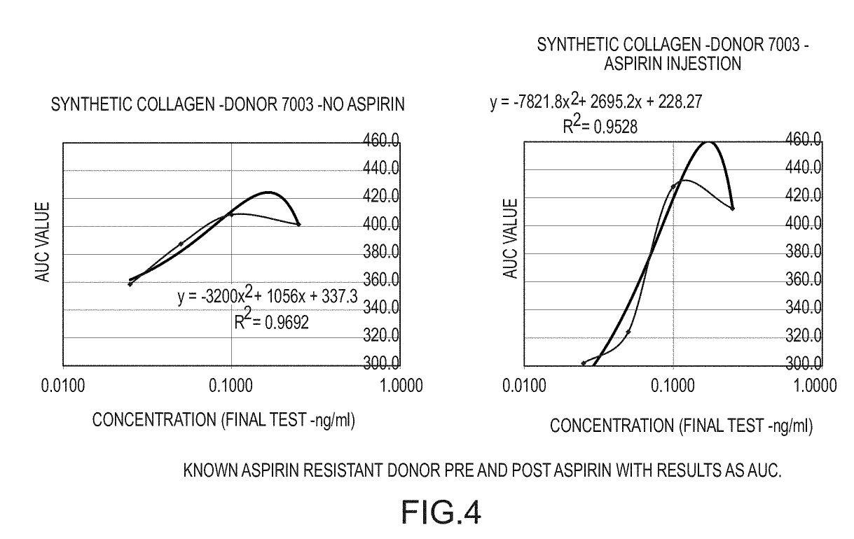

FIG. 6 provides the test results shown as AUC on a known normal/average aspirin responder using Chrono-Log (Horm) collagen before and after aspirin ingestion. This figure shows that the Chrono-Log (Horm) collagen is insensitive to aspirin because the pre-aspirin and post aspirin AUCs are very similar and would be difficult if not impossible to distinguish. This insensitivity to aspirin in one reason why Chrono-Log (Horm) collagen could not be used to determine the combined effect of aspirin and an anti-platelet medication on platelet activity/aggregation. Further, the non dilutability of biological collagen limits its sensitivity and usefulness in assessing anti-platelet medication effectiveness.

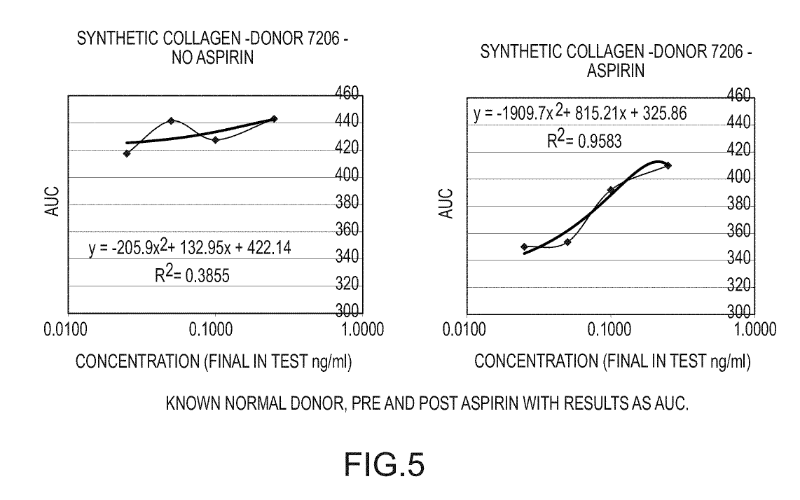

FIG. 7 provides a block diagram of a dual anti-platelet medication therapy test.

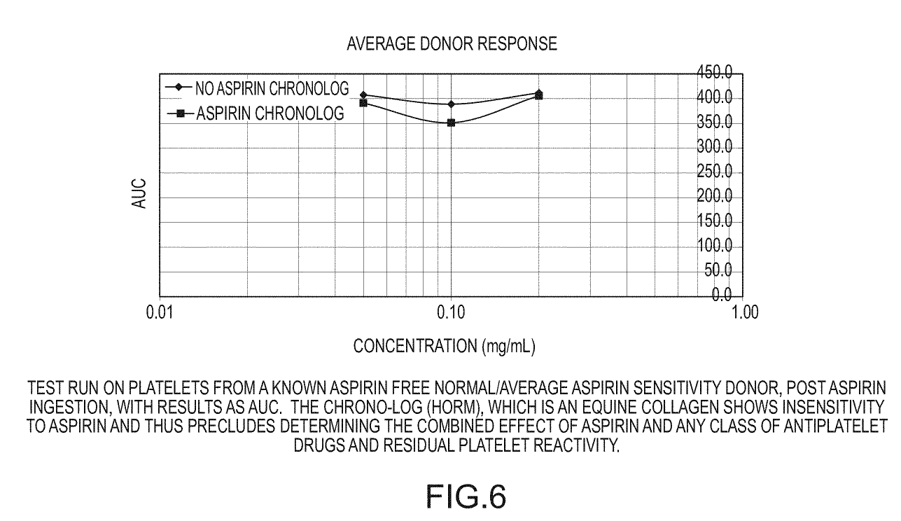

FIGS. 8-13 show the results of tests where synthetic collagen was used to detect anti-platelet activity of various anti-platelet medications. In these tests, light transmission assays using Bio/Data's PAP 8E platelet aggregometer was used. The ICHOR II impedance cell counter was used to count the platelets. See Example 1.

FIG. 8 shows the effect of ticagrelor on agonist-induced platelet aggregation.

FIG. 9 shows the effect of ticagrelor on agonist-induced platelet aggregation in aspirinized plasma.

FIG. 10 shows the effect of cilostazol on agonist-induced platelet aggregation.

FIG. 11 shows the effect of cilostazol on agonist-induced platelet aggregation in aspirinized plasma.

FIG. 12 shows the effect of abciximab on agonist-induced platelet aggregation.

FIG. 13 shows the effect of abciximab on agonist-induced platelet aggregation in aspirinized plasma.

FIG. 14 shows biological collagen at 5 and 2 μg/mL. In using the whole blood mode of the Chrono Log aggregometer, a test measuring platelet aggregation on whole blood (impedance aggregation), at 5 μg/mL, the biological collagen gets a response. At 2 μg/mL, there is no response. As an aside, even though called “whole blood mode” by the manufacture, most often whole blood is actually diluted whole blood (usually a 1:1 or greater dilution).

FIG. 15 shows that synthetic collagen can be diluted from 100 ng/mL to 12.5 ng/mL and still elicit the same response using the whole blood mode of the Chrono Log aggregometer, a test measuring platelet aggregation on whole blood (impedance aggregation).

### DETAILED DESCRIPTION OF THE INVENTION

The present invention provides dual anti-platelet therapy tests (“DAPT™”). The DAPT™ test is a unique, quantitative, functional test, based on different synthetic collagen concentrations, that measures the patient's response to different classes of anti-platelet medications administered concurrently with aspirin, as well as the combined residual platelet reactivity of the aspirin and anti-platelet medication inhibited patient platelets. The test results provide the physician with information about the patient's response to the combination of aspiring and anti-platelet medication as well as discreet and combined residual platelet activity. The dual anti-platelet therapy tests of the present invention use platelet aggregation assays to measure platelet aggregation or inhibition of platelet aggregation, which include, but are not limited to light transmission aggregometry (LTA) (which uses platelet rich plasma (“PRP”)); flow cytometry (which uses whole blood); whole blood impedance aggregometry.

The present invention also provides a method for testing a patient's compliance with a dual therapy regimen by monitoring the patient's platelet response over time. Compliance with the dual therapy means both taking both the medications (aspirin and an anti-platelet medication), and taking them at the proper time to maintain its risk-reducing anti-platelet effect. It is also known that tailoring the second drug (the anti-platelet medication) in dual therapy is important because the pathways that process the anti-platelet drugs also process other drugs. For example, in a patient on dual anti-platelet therapy in which one drug is ticagrelor, the plasma level of statins is increased and may be above the therapeutic level. High dose statin therapy avoidance is recommended by the FDA. Other drugs, such as digoxin, must be regularly monitored when a patient is on dual antiplatelet therapy. Incidentally, the aspirin dose must not go over 100 mg, or ticagrelor effectiveness decreases and MACE risk increases.

Residual platelet activity is the activity (functionality) of the platelets after they have been exposed to the dual anti-platelet medication therapy. No therapeutic dose will impair 100% of the platelets, nor would any combination reach 100% (nor would this be desirable). In this case the major adverse clinical event (MACE) would be severe bleeding. However, the reactivity of these non-impaired platelets is a key factor in understanding the individual's complete platelet response and MACE risk. For instance if the dual anti-platelet medication therapy were to impair 80% of the platelets (keep 80% platelets from aggregation), there is still 20% of the individual's platelets that could cause thrombotic risk if they were to be extremely active or alternatively, could pose a risk of bleeding to death if the platelets were not active and would not aggregate (and thus would not clot).

The present invention provides a method for determining an individual's functional response to an anti-platelet medication when the individual is on, or is a candidate for a dual anti-platelet medication therapy regimen. A dual anti-platelet therapy regimen means an anti-platelet aggregation regimen involving the administration of a low or patient specific dose of aspirin and at least one other anti-platelet medication, typically a first generation thienopyridine such as clopidogrel or increasingly, ticagrelor—a cyclopentyltriazolopyrimidine.

Aspirin is a common drug whose active ingredient is acetylsalicylic acid (ASA or ASS). It is a weak acid that is absorbed across the mucosal lining of the stomach and small intestine. After absorption, ASA is (metabolized) hydrolyzed to acetic acid and salicylic acid. In most individuals, aspirin causes inhibition of platelet aggregation and thus, aspirin is used in many therapies where it is desired to minimize platelet aggregation. These individuals are sometimes referred to as normal or average aspirin sensitive.

However, in some individuals, even after taking aspirin, the platelets will still aggregate, and hence aspirin therapy would not be beneficial or at the very least would not be beneficial alone. These individuals are often referred to as aspirin resistant or aspirin non-responsive, or high on treatment platelet reactivity (platelets are still highly reactive while patient is on therapy). In this situation, it is important for the physician to know if the patient is compliant or non-compliant in their therapy regimen rather than being non-responsive to aspirin. Several studies report patient noncompliance to range from 30-70%. Noncompliance, which is currently only documented by patient interviews, may be a major reason for outcome variability.

In yet other individuals, even very small doses of aspirin cause a severe inhibition of platelet aggregation that could lead to serious bleeding issues. These individuals can be called aspirin hypersensitive. For these individuals aspirin therapy may cause more harm than good because of the increased bleeding risk. In other individuals, aspirin administration causes a life threatening anaphylactic reaction. These individuals are said to be aspirin intolerant. Thus, it is very desirable to be able to test a patient/donor for their response to aspirin to see what effects the aspirin will have on the patient's platelet aggregation to determine whether the aspirin therapy will be useful for preventing unwanted platelet aggregation or whether aspirin therapy should be avoided altogether or perhaps instead be used with another drug to enhance the aspirin effect.

Low-dose aspirin (81 mg) is the most common dose used as a preventative regimen against a heart attack or a stroke. However, the dose for daily aspirin can range from 81 mg to 500 mg. Tablets marketed as “low-dose aspirin” contain 81 mg aspirin. One adult-strength tablet contains about 325 mg aspirin, and “advanced” extra strength contains 500 mg. Patient specific aspirin doses can vary from 81 mg to 500 mg. Resistance or insensitivity can occur at any of these doses.

Antiplatelet medications are known and include, but are not limited to, abciximab (Reopro®), anagrelide (Agrylin®), apixoban (Eliquis®), clopidogrel bisulfate (Plavix®), eptifabatide (Integrilin®), tirofiban (Aggrastat®), dipyridamole/aspirin (ASA) (Aggrenox®), cilostazol (Pletal®); dipyridamole (Persantine®), ticlopidine (Ticlid®), ticagrelor (Brilinta®), Aloxiprin (aluminum acetylsalicylate), Carbasalate calcium (mixture of calcium acetylsalicylate and urea), Cloricromen, Clorindione, Ditazole, Indobufen, Picotamide, Ramatroban, Terbogrel, Terutroban, and triflusal, as well as those in similar drug classes that are in various stages of development like cangrelor, elinogrel, prasugrel, and others. Aspirin is also considered an anti-platelet medication. For purposes of this application, when referring to a “dual therapy” it is meant as a therapy that includes aspirin and an anti-platelet medication (that is not aspirin).

The methods of the present invention are able to test the ability of the individual's platelets to aggregate after the individual has ingested an anti-platelet medication and aspirin. In addition, methods of the invention provide assays that “discount” or “ignore” the effect of the aspirin on platelet aggregation, but still measure the effect of the anti-platelet medication on the platelet aggregation. Further, the invention provides assays that “discount” or “ignore” the effect of the anti-platelet medication, but still measure the effect of the aspirin on platelet aggregation.

In most individuals, various anti-platelet medications will cause a variable degree of inhibition of platelet aggregation. These individuals are sometimes referred to as having an average/normal response or “low on therapy platelet reactivity.” However, in some individuals, even after taking the standard dose of anti-platelet medication, the platelets will still aggregate, and hence the particular anti-platelet medication would not be beneficial, but rather detrimental since the thrombotic risk would not be controlled, reduced or evident to the clinician. These individuals are often referred to being non-responsive. In such individuals, the clinician might change medication doses, try another drug class or even add a third drug to the anti-platelet regimen if the bleeding risk is reasonable. In yet other individuals, even very small doses of anti-platelet medication causes a severe inhibition of platelet aggregation that could lead to bleeding issues, which in some cases are life threatening or life ending. These individuals can be called hypersensitive. For these individuals a particular anti-platelet medication therapy may cause more harm than good because of the known bleeding risk attendant to the use of these types of medications. Thus, it is very desirable to be able to test a patient/individual for their response to a particular anti-platelet medication to see what effects the anti-platelet medications will have on the patient's platelet aggregation and residual platelet reactivity to determine whether the anti-platelet medication therapy will be useful for preventing unwanted platelet aggregation or whether the particular anti-platelet medication therapy should be avoided because of bleeding, interference with other drug therapies, or other serious risks.

In addition, the tests can be used to monitor patient compliance in taking the prescribed anti-platelet medication. Non-compliance has been identified in multiple studies as a significant occurrence and carries a very high risk for the patient. The preceding is the basis for the increasing shift to a personalized medicine focus, a key element of selecting the right drug or combination of drugs for the individual patient. The DAPT meets this currently unmet need.

Embodiments of the invention can test for platelet aggregation using methods known in the art, including, but not limited to flow cytometery and light transmission aggregometry (LTA) and whole blood impedance aggregometry. Flow cytometry uses whole blood and can be used to detect platelet aggregation. Light Transmission Aggregometry (LTA) studies, which are known as the “gold standard” in testing platelet aggregation. Light Transmission Aggregometry (LTA) is an in vitro diagnostic functional assay that measures quantitatively multiple optical parameters from changes in light transmission through Platelet Rich Plasma (PRP) following the addition of an agonist (such as, collagen, ADP, epinephrine, Ristocetin, Arachidonic Acid, thrombin and TRAP) (e.g. more light passes through a sample where there has been platelet aggregation as compared to a sample with no platelet aggregation). Often a blank is used for the comparison, which is the donor's plasma with no platelets present, which is often called “platelet poor plasma” (“PPP”).

An agonist is a material that when added to platelet rich plasma, causes the platelets to aggregate. In the methods of the present invention the agonist is synthetic collagen. In LTAA, the PRP is usually stirred in a cuvette at 37° C., and the cuvette sits between a light course and a photocell. After an agonist is added to platelet rich plasma (PRP), the platelets aggregate and absorb less light, so the light transmission increases and is detected by the photocell.

LTAAs generate data in the form of aggregation patterns. The LTAA generates parameters plotted on an x/y grid. The x axis is usually a linear time base (typically—minutes). The y axis is a logarithmic scale based upon light transmittance. This light transmittance is equated to percent (%) aggregation.

As the LTAA pattern or curve is generated (primary data), various derived measurements are calculated, including Slope (Sa); Maximum Aggregation; Final Aggregation; Area Under the Curve (AUC), Area Under the Slope (AUS) and others. In some embodiments of the present invention AUC is a preferred measurement aspect, because it appears more sensitive than the others. Slope of aggregation (Sa) is a measurement of the rate at which the reaction is proceeding. Dilution profile (DUP) is an incremental change in concentration of the reactants in a test mixture. In collagen testing, the DUP is comprised of the changes to the concentration of the collagen reagent used. Other dilution profiles may be defined and used in analyses. Slope of the dilution Profile (Sd) is generally the regression analysis of the change in concentration. Slope of the reaction profile (Sr) is generally the regression analysis of the change of reaction to change of dilution. The regression analysis may be linear, polynomial or other models. Area under the curve (AUC) is a receiver operating curve that is the calculated graphical volume from the start of the reaction to the end of the reaction as defined by the aggregation and slope of aggregation (Sa). The use of the AUC parameter increases the sensitivity of the DAPT assay(s). This may be considered as the “Power” generated by the reaction.

The present invention utilizes synthetic collagen, which it turns out is much more sensitive, potent, predictive and precise than biological collagen, and further is dilutable, which allows extremely low amounts of synthetic collagen to be used. Using synthetic collagen, the methods of the present invention are able measure the degree to which the patient's platelets resist aggregation after the patient has ingested aspirin and an anti-platelet medication. This is a key element, which permits the clinician to accurately assess thrombotic risk avoid MACE and improve patient outcome. In addition, as will be described below in more detail, because the synthetic collagen is dilutable and can be used at many different concentrations, (thereby the right concentration for maximum sensitivity) the tests can be manipulated using different concentrations of the collagen to test for residual platelet activity, to test the effect the anti-platelet medication is having on platelet activity, and to test the effect that the aspirin is having on platelet activity.

Further, synthetic collagen provides a means of quantitatively assessing residual platelet reactivity, which is the key indicator of prognostic risk, and is, therefore, more useful information than the currently available, qualitative and highly variable parameter called platelet inhibition. It is important to note that inhibition of aggregation does not equal residual platelet reactivity. Until recently, “platelet inhibition” was the global term and test parameter that was accepted for understanding how platelets behaved when exposed to an anti-platelet drug. The percent aggregation or percent inhibition of aggregation was simply adopted because that is how platelet aggregation was reported. So, inhibition was simply the difference between the patient's original aggregation result and the post treatment result. If the patient's original aggregation result was 82% and the post treatment aggregation result was, 23%, the percent inhibition was reported as 59%. It was then assumed that the other 41% of the platelets were not inhibited. The target was to get the percentage of inhibition between 60 and 80% because that would mean the patient would neither clot nor bleed (ideal outcome). However, it has now been determined that this simplistic construct or understanding does not tell the whole story and thus not work because it does nothing to aid in the understanding of the residual platelet reactivity (that is, the reactivity of the platelets that did not respond to the ingested medicine). There simply is no direct, measurable or predictable relationship between percent inhibition and residual platelet reactivity.

Physicians have discovered that patient outcomes to various treatments would vary even when they shared the same percent inhibition. For example, two patients who showed 41 percent inhibition (and thus had 59% aggregation) in an LTA might respond very differently (one may still have problems with unwanted clotting and the other may have problems with bleeding). This observation has slowly led to the realization that although the percent inhibition number (the number of remaining or uninhibited platelets) could be identical, the patient responses could be very different. This has recently spawned the use of terms such as hyper and hypoactive, hyper and hypo-responders, platelet reactivity, residual platelet reactivity, and High on Treatment Platelet Reactivity.

The point is that percent inhibition is not the same as residual platelet reactivity. It is clear that platelet response is the measurable effect of a challenge on platelet function, i.e., how a particular drug interferes with a platelet function pathway (platelet and metabolic genetics, drug action and pharmacokinetics, and other factors all come in to play for residual platelet reactivity). The present invention can measure residual platelet reactivity, which is a combination of three things wrapped up into basically a single measurement. The first component is a dose response based on the primary drug (e.g. an anti-platelet medication) to show what portion of the patient's platelets are rendered partially or non-functional based on the that particular patient's individual response to that drug and dose. The second component relates to how reactive the remaining platelets are. These platelets, like the ones that are inhibited, could be hyperactive, hypoactive or anywhere on the continuum between those two points to a different medicine. The third component provides information about the effect of the second drug (e.g. aspirin), which has all the same considerations of the first drug.

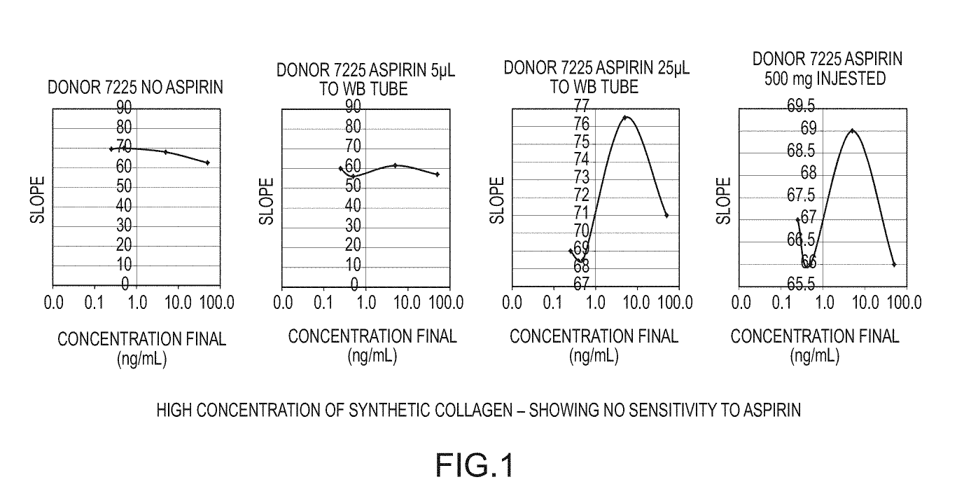

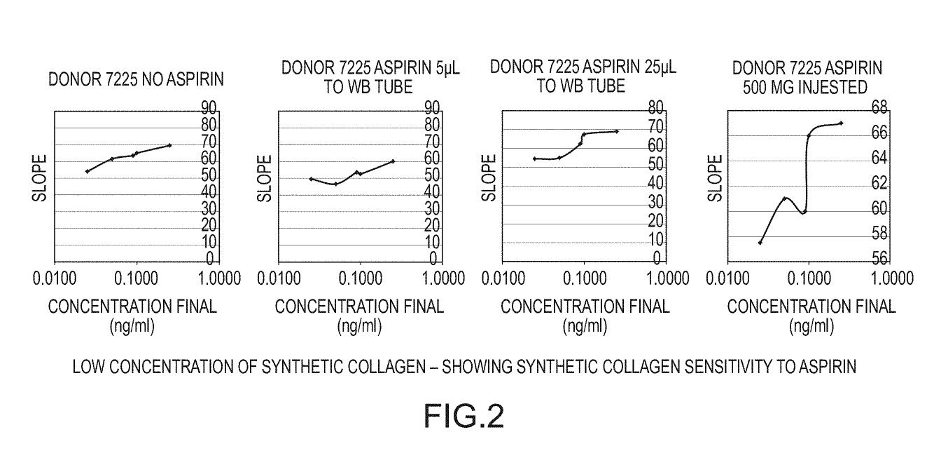

Synthetic collagen has a unique ability to be insensitive to aspirin at certain concentrations, which allows for the assessment of the second anti-platelet medication, along with its residual platelet reactivity and yet can be used at extremely low concentrations to allow assessment of aspirin sensitivity alone (see FIGS. 1 and 2) and further, at other concentrations, can be used to assess residual platelet activity (the activity of the platelets remaining in response to both the anti-platelet medication and the aspirin. FIG. 1 shows no aspirin sensitivity when the synthetic collagen concentration is high. FIG. 2 shows aspirin sensitivity when the synthetic collagen is low. The insensitivity to aspirin is a key factor in measuring the second anti-platelet drug while the sensitivity to aspirin is needed to assess the residual platelet activity (the activity remaining despite the separate effects of the two drugs).

In one aspect, the synthetic collagen is used at a concentration that causes the test to ignore or discount the effect that that aspirin might be having on platelet activity, but still allows one to measure the effect of the anti-platelet medication on platelet activity. In this aspect, the concentration is greater than 40 ng/mL and preferably greater than 50 ng/mL.

In another aspect, the synthetic collagen is used at a concentration that causes the test to ignore or discount the effect that the anti-platelet medication might be having on the platelets but still allows one to measure the effect of the aspirin on platelet activity.

In another aspect, the amount of synthetic collagen is used at a concentration that tests the residual activity of the platelets after being exposed to the aspirin and the anti-platelet medication. In this aspect, the concentration of synthetic collagen is between about 25 ng/mL to about 35 ng/mL.

There are ranges of platelet aggregation that occur after an individual consumes an anti-platelet medication and these ranges can be used to characterize an individual as having a hypersensitive response, a normal/average response, or having a non-response to the anti-platelet medication. Different individuals respond to a particular anti platelet medications differently, so the present invention provides a way of measuring the response to the anti-platelet medication as well as residual platelet reactivity. If the individual does not respond to the medication as desired, the physician can then change the dose, prescribe a different anti-platelet medication or even add a third drug.

The present invention also provides embodiments that capitalize on the repeatability and sensitivity of the synthetic collagen as well as its ability to be reproducibly diluted over a range of concentrations and thus, employs multiple dilutions of synthetic collagen (referred to herein as “dilution profiles”) to aid a physician in determining not only whether an individual is sensitive to the anti-platelet medication and/or aspirin, but to further understand an individual's anti-platelet medication and/or aspirin sensitivity status (e.g. the degree to which an individual is anti-platelet medication and/or aspirin sensitive, non-responsive or hypersensitive). This information may be useful for the physician to determine an appropriate dose (patient specific) of anti-platelet medication and/or aspirin in the prescribed therapeutic regimen, or perhaps whether a second or third therapeutic medicine is required, or whether to consider abandoning the use of the anti-platelet medication and/or aspirin altogether for an alternative therapy.

Further capitalizing on the sensitivity of synthetic collagen and further using the dilution profile concept, the present invention also provides embodiments where an individual's anti-platelet medication and/or aspirin sensitivity can be predicted even before the donor ingests the anti-platelet medication. Aspirin and anti-platelet medication non-responder (resistant) donors have a distinct response (referred to herein as the “bounce back”) to LTAAs run over varying concentrations of synthetic collagen, which can be used to diagnose a donor's response to the aspirin and the anti-platelet medication. This and other embodiments are discussed more fully herein below.

As mentioned above, LTAAs use Platelet Rich Plasma (PRP), which is prepared from properly anti-coagulated whole blood. The individual's blood is collected and spun down to obtain the PRP. Since platelets are very sensitive and can be readily activated during the preparation of PRP, the individual's blood is usually collected in a tube containing a particular anticoagulant. For example, venous blood is obtained and collected into 3.2% sodium citrate in a ratio of 1:9 (1 part anticoagulant to 9 parts blood). Whole blood samples should be processed within 4 hours of collection and blood samples for platelet aggregation testing must be stored at room temperature as cooling the platelets can lead to activation and erroneous test results. PRP is usually prepared by centrifugation at 20° C. for 10-15 minutes at 150-200 g, or, with platelet function centrifuge such as the PDQ companion centrifuge for the PAP 8E, PRP can be prepared in under 4 minutes. The PRP is carefully removed and placed into a stoppered plastic tube. PRP must be stored at room temperature.

Platelet poor plasma (PPP) can be then prepared by further centrifugation of the remaining plasma at 2700 g for 15 minutes. Platelet poor plasma (PPP) contains no platelets or other cellular material, and is often used as a blank in LTA sample analyses. By using the PDQ platelet function centrifuge, this time can be reduced to about three minutes. In certain embodiments a special centrifuge that can repeatedly generate PRP and PPP in about 5 minutes instead of the typical 45-60 minutes is employed. This makes the LTAA even more practical for emergency and critical care situations or for a high throughput clinical setting. In addition to the rapid sample preparation, the use of certain concentrations of synthetic collagen can be used to generate the patient's global residual platelet reactivity on an emergency basis.

Addition of a platelet agonist to the PRP usually leads to platelet activation, and leads to a change in their shape from discoid to spiny spheres, which is associated with a transient increase in optical density. Exceptions to this are epinephrine in which there is no shape change, and ristocetin, which causes platelet agglutination rather than aggregation, i.e. there is no binding of fibrinogen. Agonists are usually classified as strong agonists or weak agonists. Strong Agonists (e.g. Collagen, thrombin, TRAP, high concentration ADP, and U46619 (an analog of TxA2)) directly induce platelet aggregation, TxA2 synthesis and platelet granule secretion. Weak Agonists (e.g. low concentration ADP & epinephrine) induce platelet aggregation without inducing secretion.

In general, LTAs are performed at 37° C. The aggregometer is calibrated by: 1) a cuvette containing PRP, which equates to 0% light transmission; and 2) a second cuvette containing PPP, which equates to 100% light transmission. Since platelets will normally only aggregate if they are activated (with an agonist) and in contact with each other, they must be stirred whilst testing is taking place. Absence of stirring will lead to an absence of, or at least a significant reduction in, aggregation.

The present invention utilizes synthetic collagen as the agonist instead of collagen obtained from biological sources in the light transmission aggregometry (“LTA”). The use of synthetic collagen provides many unexpected benefits over the use of biological collagen, which is described herein.

In certain embodiments, Bio/Data's PAP 8E LTA is employed (See U.S. Pat. No. 7,453,555) as the LTA used in the LTAAs of the present invention.

Normally a test for spontaneous platelet aggregation (“SPA”) is performed. SPA is rare in healthy individuals, but occurs in people with hyperactive platelets and is also recognized as an independent marker for some pro-thrombotic conditions, in some cases of von Willebrand Disease (vWD), in some patients with diabetes, in some lipid disorders and in a variety of other disorders. The presence of SPA is tested by placing undiluted PRP in the aggregometer and stirring for 15 minutes. In cases of SPA, dilution of the PRP may abolish this and if the platelet count remains >200×10⁹/L then aggregation testing can proceed.

In general, about 225 μL of PRP is added to the aggregometry test cuvette and warmed at 37° C. Then 25 μL of the agonist is added and the response recorded. The typical readouts or responses recorded include primary aggregation (“PA”) (which usually provides a value indicating the amount of aggregation), primary slope (“PS”)(which usually provides a value relating to the speed of aggregation) and area under the curve (“AUC”)(which generally provides a value relating to a combination of PA and PS). Aggregometry analyzers used in the field typically will provide these readouts along with a pictorial graph of the aggregation. Each aggregometer or system calculates the values a bit differently and may use a proprietary formulae embedded in the system software.

For example, % maximal aggregation may be calculated by measuring the distance between the baseline [0% aggregation−platelet rich plasma] and platelet poor plasma [100% aggregation] [Y] and dividing this number by the maximal aggregation [X]. So if the Y=100 mm and X=89 mm then percentage maximal aggregation=X/Y=89%.

In the methods/assays of the present invention, instead of having the patient ingest aspirin and thereafter take a blood sample, a sample of blood can be taken before any aspirin is ingested and the sample can be “aspirinated” (or “aspirinized”)(that is, an aspirin solution (may also be the lysine salt of aspirin or Aspisol®) is added to the PRP and then tested). It has been determined that in the all of the methods/assays of the present, either the patient can ingest the aspirin or the sample can be aspirinated. This can speed up the testing because the patient does not need to ingest the aspirin and have time pass to allow the aspirin to get into the patient's system. Instead, the blood is drawn and a PRP sample is obtained, and one part is aspirinated and the other part is not, thus also allowing the two samples to be tested side by side.

A. Testing for Platelet Sensitivity to Anti-Platelet Medication in an Individual on Dual Anti-Platelet Medication Therapy (Ignoring the Effect of Aspirin at this Stage of the Assay, but can be Measured at Another Stage)

One embodiment of the present invention provides tests that can determine the individual's platelet sensitivity to an anti-platelet medication when the individual is on a dual anti-platelet medication therapy (i.e. on aspirin and an anti-platelet medication). In these embodiments the final in-test concentration of synthetic collagen that is used tests the ability of the platelets to aggregate in the presence of an agonist (synthetic collagen) and is at a concentration where the test ignores the effect of aspirin on platelet aggregation but still measures the effect of the anti-platelet medication on the platelet aggregation. Preferably, in these embodiments the final in-test concentration of synthetic collagen used ranges from about 50 ng/mL to about 500 ng/mL; or is >40 ng/mL; or is >50 ng/mL; or ranges from about 40 to about 500 ng/mL; or ranges from about 40 to about 400 ng/mL; or ranges from about 40 to 300 ng/mL; or ranges from about 40 to about 200 ng/mL; or ranges from about 40 to about 100 ng/mL; or ranges from about 40 to about 90 ng/mL; or ranges from about 40 to about 80 ng/mL; or ranges from about 40 to about 70 ng/mL; or ranges from about 40 to about 60 ng/mL; or ranges from about 50 to about 400 ng/mL; or ranges from about 50 to about 300 ng/mL; or ranges from about 50 to about 200 ng/mL; or ranges from about 50 to about 100 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

This method involves performing one or more platelet aggregation assays, such as light transmission assays or flow cytometry whereby a first platelet rich sample or whole blood sample is obtained from an individual and is combined with synthetic collagen to form a first treated sample. For the first treated sample, the individual has not ingested the anti-platelet medication for a time period of about 24 hours, preferably 72-96 hours. The idea is to make sure that the individual will not have any anti-platelet medication in his system to affect the platelet aggregation tests. The first treated sample is tested/measured for platelet aggregation to get a first readout to determine the individual's baseline level of platelet aggregation in the absence of ingested anti-platelet medication. If LTA is used, the sample is placed into a LTA aggregometer and light transmission through the first treated sample is obtained to get the first readout.

In certain embodiments, an initial LTAA or other platelet aggregation assay may be performed to check for spontaneous aggregation to test for whether the platelets have any inherent hyperactivity.

Then the individual is given the anti-platelet medication (presumably the individual is already on aspirin therapy and has ingested aspirin) and a time period sufficient to allow the anti-platelet medication to be metabolized (e.g. at least about 2 hours to about 16 hours) is allowed to pass before a second platelet rich plasma sample is obtained from the donor. Another platelet aggregation assay such as LTAA is performed on the second platelet rich plasma sample by treating it with synthetic collagen to form a second treated sample. Platelet aggregation is measured to obtain a second readout. If LTAA is the assay used to measure platelet aggregation, light transmission through the second sample is measured to obtain the second readout.

It is preferred that the same type of platelet aggregation assay is used throughout the process. For example, if LTAA is used for the first treated sample, then preferably LTAA is used for the second treated sample.

The baseline level readout of platelet aggregation in the absence of ingested anti-platelet medication is compared with the second treated sample readout (obtained after the anti-platelet medication ingestion) and the results of this comparison will determine the individual's anti-platelet medication response status. For example, if the individual shows a significant reduction in platelet aggregation after the anti-platelet medication ingestion (in the second sample) as compared to the baseline sample, then the individual may be characterized as normal or having an average anti-platelet medication sensitivity. If the individual shows very little difference in the platelet aggregation after taking the anti-platelet medication (i.e. the platelets still aggregated after the individual ingested the anti-platelet medication), then the individual may be characterized as being anti-platelet medication non-responsive. If the individual showed an almost complete lack of platelet aggregation after ingesting the anti-platelet medication, then the individual may be characterized as being anti-platelet medication hypersensitive, which itself is a very high risk state for the patient.

In certain embodiments, the test uses LTAA for platelet aggregation measurements. The readout from the LTAA may be slope, primary aggregation, area under the curve, lag phase disaggregation or a combination thereof.

For example, when using the Bio/Data's PAP 8E aggregometer, and when using an “in-test” concentration of 50 ng/mL of synthetic collagen, for an anti-platelet medication hypersensitive individual, the baseline for PA will range from 40% to 100%. The baseline for PS will range from 20 to 60. The baseline for AUC will range from 300 to 700. After the anti-platelet medication, the AUC will range from 100 to 400. PS, PA and LP, will be different from their respective baselines. The anti-platelet medication sensitive and the anti-platelet medication non-responders will show differences from baselines; sensitive individuals will show less aggregation. In certain embodiments, an algorithm that combines and categorizes this data, into an actionable form useful to the physician is.

In these tests, the individual may or may not have also consumed aspirin for the test. In one embodiment, the individual does not consume aspirin before the first platelet sample is obtained for the baseline readout, but then ingests aspirin before the second assay is run. In another embodiment, the individual was already on aspirin therapy and had ingested aspirin before the baseline readout was obtained and continued taking the aspirin during the testing. Since this test uses synthetic collagen at a range where it is insensitive to the effects of aspirin on platelets, it does not matter for the test results if the individual consumes aspirin. The beauty of the test is that any inhibition of platelet aggregation seen in the test occurs because of the effect of the anti-platelet medication on the platelet activity and not the aspirin.

In another embodiment, no baseline readout is obtained. In this situation, it may be desirable, but is not necessary, to use a previous test run for this individual before the individual started the dual anti-platelet medication therapy as the baseline readout. In the situation where no baseline was obtained, the assay involves performing one or more tests, such as light transmission assays, whereby a first platelet rich sample is obtained from an individual and is combined with synthetic collagen to form a treated sample. The sample is tested and in the case of LTAA is placed into a LTA aggregometer and the light transmission through the treated sample is obtained to get a readout to determine the individual's level of platelet aggregation. In certain embodiments, an initial test may be performed to check for spontaneous aggregation. The results of this treated sample assay are used to determine the individual's anti-platelet medication response status. For example, if the individual shows a significant reduction in platelet aggregation, then the individual may be characterized as normal or having an average anti-platelet medication sensitivity. If the individual shows very little inhibition of platelet aggregation after taking the anti-platelet medication (i.e. the platelets still aggregated after the individual ingested the anti-platelet medication), then the individual may be characterized as being anti-platelet medication non-responsive. If the individual showed an almost complete lack of platelet aggregation after ingesting the anti-platelet medication, then the individual may be characterized as being anti-platelet medication hypersensitive.

If the test used LTAAs then the readout of platelet aggregation from the LTAA may be slope, primary aggregation, area under the curve, lag phase, disaggregation, final aggregation or a combination thereof.

### B. Testing for Platelet Sensitivity to an Anti-Platelet Medication in an Individual on Dual Anti-Platelet Medication Therapy Using a Dilution Profile (Ignoring the Effect of Aspirin on the Platelets)

In another embodiment, a dilution profile of synthetic collagen is utilized across a number of different platelet aggregation tests run on an individual's whole blood or PRP sample. In this embodiment of the invention, more than two reactions (more than just the baseline test before the anti-platelet medication ingestion and the test after the anti-platelet medication) are run. A series of platelet aggregation tests, such as a series of LTAAs, are run using multiple differing amounts of synthetic collagen. This is referred to herein as the “dilution profile assays” or “dilution profiles.” In this embodiment, multiple different whole blood or PRP samples are obtained from the individual before the anti-platelet medication ingestion (to obtain a baseline dilution profile) and after the anti-platelet medication ingestion (to obtain a post anti-platelet medication dilution profile). Each individual pre-anti-platelet medication platelet sample is mixed with a different amount of synthetic collagen and a platelet aggregation assay, such as LTAA, is performed on each sample to obtain a baseline dilution profile over the range of concentrations. Then the individual is given the anti-platelet medication and sufficient time is allowed to pass to ensure the anti-platelet medication has been metabolized. Multiple whole blood or PRP samples are obtained from the individual post anti-platelet medication ingestion and mixed with different amounts of synthetic collagen. Platelet aggregation tests, such as LTAAs, are performed on each different sample to obtain a post-anti-platelet medication dilution profile. The same concentrations of synthetic collagen that were used in the pre-anti-platelet medication baseline platelet aggregation tests are preferably used in the post-anti-platelet medication platelet aggregation tests. The results are analyzed and the change in platelet aggregation between the pre- and post-anti-platelet medication tests as well as the change of aggregation over the differing amounts of synthetic collagen are studied to determine the individual's anti-platelet medication sensitivity response (whether the individual is anti-platelet medication hypersensitive, average anti-platelet medication sensitive or anti-platelet medication non-responsive and the degree of sensitivity therein).

If the tests utilized LTAAs then the PA, PS or AUC or a combination thereof between the pre- and post-anti-platelet medication LTAs, as well as changes in the PA, PS or AUC or a combination thereof over the differing amounts of synthetic collagen, are studied (often using an algorithm that categorizes the data or information and reports the data) to determine the individual's anti-platelet medication sensitivity response (whether the individual is anti-platelet medication hypersensitive, average anti-platelet medication sensitive or anti-platelet medication non-responsive and the degree of sensitivity therein). In certain embodiments, the results are characterized using the aggregometer's proprietary algorithm embedded in system software, which makes the analysis easier for the diagnostician to understand and make appropriate clinical decisions.

In other embodiments, the pre-anti-platelet medication baseline is established with one platelet aggregation test (such as an LTAA) performed using one concentration of synthetic collagen (such as 50 ng/mL) in the LTAA on an individual pre-anti-platelet medication platelet sample, whereas multiple different concentrations of synthetic collagen are still used in different post anti-platelet medication platelet aggregation tests, such as LTAAs, to create the post-anti-platelet medication dilution profile. In this case, the results are analyzed and the change in platelet aggregation seen in the different amounts of synthetic collagen are studied, and compared against each other as well as to against the baseline (pre-anti-platelet medication) to determine the donor's anti-platelet medication sensitivity response (whether anti-platelet medication hypersensitive, normal/average anti-platelet medication sensitive or anti-platelet medication non-responsive and the degree of sensitivity therein).

When the platelet aggregation tests use LTAAs, the PA, PS, AUC, or a combination thereof from differing amounts of synthetic collagen are studied, and compared against each other as well as to against the baseline (pre-anti-platelet medication) LTAA to determine the donor's anti-platelet medication sensitivity response.

In other embodiments, a pre-anti-platelet medication baseline or pre-anti-platelet medication dilution profile is not obtained. This may be useful in the emergency clinical setting when it is not feasible to obtain a pre-anti-platelet medication baseline or whether one cannot determine from the patient whether he or she has been on anti-platelet medication therapy. In this embodiment, multiple different platelet rich plasma samples or whole blood samples are obtained from the individual and each are mixed independently with a different synthetic collagen concentration to obtain multiple different treated samples for the dilution profile tests. Platelet aggregation tests, such as LTAAs, are performed for each of these samples to obtain a dilution profile over the range of different concentrations. The data is obtained and measured.

When the platelet aggregation tests are LTAAs, the AUC, PA, and/or PS or combination therefore are obtained and analyzed over the different ranges of synthetic collagen. In certain embodiments, the results are analyzed using the aggregometer's proprietary algorithm embedded in system software.

The inventors have determined that this embodiment can be used to predict the donor's platelet the anti-platelet medication response. For individuals having an average or “normal” anti-platelet medication sensitivity, when looking at the slope, percentage aggregation and/or the AUC, over the dilution profile, the slope, percentage aggregation and/or the AUC will show a corresponding decrease along with the decrease in the amount of synthetic collagen used. Thus, for example, within a given range of various dilutions of synthetic collagen, as the concentration goes down, so will the slope, percentage aggregation, and the AUC. There seems to be an almost linear decrease in slope, percentage aggregation and AUC that runs almost parallel or has almost a direct correlation with the concentration of synthetic collagen. On the other hand, for individuals who are anti-platelet medication non-responders, instead of having a slope, percentage aggregation, and/or the AUC that has a linear-like decrease that corresponds with the decrease in synthetic collagen, there is a point along the dilution profile where there is an increase in slope, an unexpected temporary increase in percentage aggregation and/or a temporary increase in AUC when there should be a decrease (because the concentration of synthetic collagen decreases). Then, as the dilution profile continues to decrease, the slope and AUC “bounce back” down to where it should be (based on the dilution of synthetic collagen) and where it was before the temporary increase and then continues along decreasing. The point at which the bounce back will occur will be at different concentrations of synthetic collagen, and will depend upon the medication class and dose, as well as the patient's metabolic and platelet receptor genetics.

Anti-platelet medication hypersensitive individuals will show increases in PA, PS and AUC compared to expected/normal results.

In certain embodiments of the invention utilizing the dilution profile concept, a series of 7, 6 or 5 different concentrations are used to develop a dilution profile, and in other embodiments, 4 different concentrations are used and yet in other embodiments, 3 or 2 different concentrations are used. Using too many different concentrations can make the test cumbersome and time consuming, whereas using too few concentrations reduces the amount of data obtained and limits the sensitivity analysis.

The range of synthetic collagen used is the dilution profile is preferably within the “anti-platelet medication sensitive range,” which is defined herein as the range of concentrations in which in an average anti-platelet medication sensitive individual the measured platelet activity/aggregation is reduced corresponding with decreasing amounts of synthetic collagen concentrations (e.g. the AUC and/or the slope decreases with the concentration of collagen).

In these embodiments the final in-test concentration of synthetic collagen used in the dilution profiles tests the ability of the platelets to aggregate in the presence of an agonist (synthetic collagen) and is at a concentration where the test ignores the effect of aspirin on platelet aggregation but still measures the effect of the anti-platelet medication on the platelet aggregation. In certain embodiments the different synthetic collagen dilution amounts comprise multiple different synthetic collagen amounts chosen from within the concentration range from about 50 ng/mL to about 500 ng/mL or the range from about 50 ng/mL to about 250 ng/mL.

For example, in certain embodiments there are seven different synthetic collagen amounts as the final “in test” concentration as follows: 500 ng/mL; 325 ng/mL; 250 ng/mL; 150 ng/mL; 100 ng/mL; 75 ng/mL; and 50 ng/mL. In other embodiments, there are different synthetic collagen amounts (2, 3, 4, 5, 6 or 7 different dilutions) each chosen from within the range of about 50 ng/mL to about 500 ng/mL or each chosen from within the range of about 50 ng/mL to about about 250 ng/mL, or chosen within the range of about 40 ng/mL to about 500 ng/mL. As some non-limiting examples, in certain embodiments, there are 7 different concentrations (50 ng/mL, 75 ng/mL, 100 ng/mL, 150 ng/mL, 250 ng/mL, 325 ng/mL, and 500 ng/mL). In certain embodiments there are 6 different concentrations (50 ng/mL, 100 ng/mL, 150 ng/mL, 200 ng/mL, 250 ng/mL, and 300 ng/mL). In certain embodiments, there are 5 different concentrations (100 ng/mL, 200 ng/mL, 300 ng/mL, 400 ng/mL, and 500 ng/mL). These values and ranges are preferably used when the platelet aggregation tests are light transmission assays and the sample is a PRP sample that is tested. However, they may also be used in other analyzers, including flow cytometers and impedance aggregometers or their equivalents.

### C. Testing for Platelet Sensitivity to Aspirin in an Individual on Dual Anti-Platelet Medication Therapy (Ignoring Effect of Anti-Platelet Medication)

One method of the present invention provides tests that can determine an individual's platelet sensitivity to aspirin when the individual is on a dual anti-platelet medication therapy. In this embodiment, the concentration of synthetic collagen is low enough to be insensitive to the effects of the antiplatelet medication on platelet activity but high enough to be sensitive to the effects of aspirin on platelet activity. In other words, in these embodiments the final in-test concentration of synthetic collagen that is used tests the ability of the platelets to aggregate in the presence of an agonist (synthetic collagen) and is at a concentration where the test ignores the effect of the anti-platelet medication on platelet aggregation but still measures the effect of the aspirin on the platelet aggregation. The concentration of synthetic collagen in this is aspect is a low range and is actually so low that a biological collagen could not be diluted to this low concentration. Preferably, in these embodiments the final in-test concentration of synthetic collagen used ranges from about 0.01 ng/mL to about 1.0 ng/mL; or ranges from about 0.1 ng/mL to about 0.5 ng/mL; or ranges from about 0.1 ng/mL to about 1.0 ng/mL; or ranges from about 0.1 ng/mL to about 1.5 ng/mL; or is from about 0.5 ng/mL or less; or ranges from about 0.5 ng/mL to about 2.0 ng/mL; or is less than 2.0 ng/mL; or is less than 10 ng/mL; or is less than 5 ng/mL. These values and ranges are preferably used when the platelet aggregation tests are light transmission assays and the sample is a PRP sample that is tested.

This method involves performing one or more platelet aggregation tests, such as light transmission assays, whereby a first platelet rich sample or whole blood sample is obtained from an individual and is combined with synthetic collagen to form a first treated sample.

For this first treated sample, the individual has not ingested the aspirin for a time period of about 24 hours, preferably 72-96 hours (presumably the patient is on an anti-platelet medication). The idea is to make sure that the individual will not have any aspirin in his system to affect the platelet aggregation tests. The sample is measured, such as by placing into a LTA aggregometer and the light transmission through the first treated sample is obtained, to get a first readout to determine the individual's baseline level in the absence of ingested aspirin. In certain embodiments, an initial platelet aggregation assay may be performed to check for spontaneous aggregation to test for whether the platelets have any inherent hyperactivity.

Then the individual's is given the aspirin and a time period sufficient to allow the aspirin to be metabolized (e.g. at least about 2 hours to about 16 hours) is allowed to pass before a second platelet rich plasma sample or whole blood sample is obtained from the donor. A platelet aggregation study, such as LTAA, is performed on the second platelet rich plasma sample by treating it with synthetic collagen to form a second treated sample. The second sample is assayed such as in an LTAA by measuring light transmission through the second treated sample to obtain a second readout.

The baseline level readout in the absence of ingested aspirin is compared with the second treated sample readout (obtained after the aspirin ingestion) and the results of this comparison will determine the individual's aspirin response status. For example, if the individual shows a significant reduction in platelet aggregation after the aspirin ingestion (in the second sample) as compared to the baseline sample, then the individual may be characterized as normal or having an average aspirin sensitivity. If the individual shows very little difference in the platelet aggregation after taking the aspirin (i.e. the platelets still aggregated after the individual ingested the aspirin), then the individual may be characterized as being aspirin non-responsive. If the individual showed an almost complete lack of platelet aggregation after ingesting the aspirin, then the individual may be characterized as being aspirin hypersensitive.

Instead of having the patient ingest the aspirin, the sample can be aspirinated (a solution of aspirin is added to the PRP sample (or whole blood sample).

If the platelet aggregation tests used LTAAs, then the readout from the LTA may be slope, primary aggregation, area under the curve, or a combination thereof. The aspirin sensitive and the aspirin non-responders will show differences from baselines; sensitive individuals will show less aggregation. In certain embodiments, an algorithm that combines and categorizes this data, into an actionable form useful to the physician is employed. During these tests the individual is also preferably taking an anti-platelet medication. Because the amount of synthetic collagen used is so low, the test is insensitive to any action of the anti-platelet medication on the platelets. This allows the tester to analyze the effects of the aspirin on platelet activity, while the individual is on the dual anti-platelet medication therapy. This can provide useful information, for example, in cases where the individual turns out to be an aspirin non-responder. In this case, the physician might take the patient off of the aspirin altogether and might prescribe a second anti-platelet medication. Or it could turn out that the patient is hypersensitive to aspirin, which may render the patient at a high risk to unwanted bleeding complications. In this case, the physician may take the patient off of the aspirin altogether and might prescribe a second anti-platelet medication.

In another embodiment, no baseline readout is obtained. In this situation where no baseline is obtained, the test involves performing one platelet aggregation assay whereby a platelet rich sample or a whole blood sample (in the case of flow cytometry) is obtained from an individual and is combined with synthetic collagen (e.g. (at a concentration ranging from about 1.0 ng/mL to about 0.1 ng/mL) to form a treated sample. The sample is measured for platelet aggregation, such as in an LTAA by placing it into a LTA aggregometer and the light transmission through the treated sample is obtained to get a readout to determine the individual's level of platelet aggregation). In certain embodiments, an initial assay may be performed to check for spontaneous aggregation. The results of this treated sample are used to determine the individual's aspirin response status. For example, if the individual shows a significant reduction in platelet aggregation, then the individual may be characterized as normal or having an average aspirin sensitivity. If the individual shows very little inhibition of platelet aggregation after taking aspirin (i.e. the platelets still aggregated after the individual ingested the aspirin), then the individual may be characterized as being aspirin non-responsive. If the individual showed an almost complete lack of platelet aggregation after ingesting the aspirin, then the individual may be characterized as being aspirin hypersensitive.

The readout from the LTAA may be Primary Slope, Primary Aggregation, Area Under the Curve, lag phase (LP), disaggregation (DA), final aggregatrion (FA) or a combination thereof.

### D. Testing for Platelet Sensitivity to Aspirin in an Individual on Dual Anti-Platelet Medication Therapy Using Dilution Profile (Ignoring Effect of Anti-Platelet Medication)

In another embodiment for testing platelet sensitivity to aspirin in an individual on a dual anti-platelet medication therapy, a dilution profile of synthetic collagen is utilized across a number of different platelet aggregation assays, such as LTAAs or flow cytometery run on an individual's PRP or whole blood sample. In this embodiment, multiple different PRP or whole blood samples are obtained from the individual before the aspirin ingestion (to obtain a baseline dilution profile) and after the aspirin ingestion (to obtain a post aspirin dilution profile). Each platelet sample is mixed with a different amount of synthetic collagen and a platelet aggregation assay, such as LTAA or flow cytometry, is performed on each sample to obtain a baseline dilution profile over the range of concentrations. Then the individual is given the aspirin and sufficient time is allowed to pass to ensure the aspirin has been metabolized. Multiple PRP or whole blood samples are then obtained from the individual post aspirin ingestion and mixed with different amounts of synthetic collagen. In certain embodiments, the samples are aspirinated instead of having the patient ingest aspirin. Platelet aggregation assay are performed on each sample to obtain a post-aspirin dilution profile. The same concentrations of synthetic collagen that were used in the pre-aspirin baseline assays are preferably used in the post-aspirin assays. Preferably the same type of platelet aggregation assay is used throughout the test. For example, flow cytometry is used to measure platelet aggregation for each sample. As another example, LTAAs are used to measure platelet aggregation for each sample.

The results are analyzed and the change in platelet aggregation as seen in changes in the PA, PS, AUC, LP, DA, or FA, or a combination thereof between the pre- and post-aspirin assays, as well as changes in the PA, PS, AUC, LP, DA, or FA, or a combination thereof over the differing amounts of synthetic collagen, are studied (often using an algorithm that categorizes the data or information and reports the data) to determine the individual's aspirin sensitivity response (whether the individual is aspirin hypersensitive, average aspirin sensitive or aspirin non-responsive and the degree of sensitivity therein). In certain embodiments the results are characterized using the aggregometer's proprietary algorithm embedded in system software, which makes the results of the analysis easier for the diagnostician to understand and make appropriate clinical decisions.

In other embodiments, the pre-aspirin baseline is established with one platelet aggregation assay performed using one concentration of synthetic collagen (such as 0.5 ng/mL in one LTAA) on a pre-anti-platelet medication individual platelet sample, whereas multiple different concentrations of synthetic collagen are still used in different post aspirin platelet aggregation assays to create the post-aspirin dilution profile assays. In this case, the platelet aggregation results are analyzed and compared against each other. If LTAAs were used as the platelet aggregation assay, the change in PA, PS, AUC, LP, DA, or FA, or a combination thereof from differing amounts of synthetic collagen are studied, and/or compared against the baseline (pre-aspirin) LTAA to determine the donor's aspirin sensitivity response (whether aspirin hypersensitive, normal/average aspirin sensitive or aspirin non-responsive and the degree of sensitivity therein).

In other embodiments, a pre-aspirin baseline or pre aspirin dilution profile is not obtained. This may be useful in the emergency clinical setting when it is not feasible to obtain a pre-aspirin medication baseline or whether one cannot determine from the patient whether he or she has been on aspirin therapy. In this embodiment, multiple different platelet rich plasma samples or whole blood samples are obtained from the individual and each are mixed independently with a different synthetic collagen concentration to obtain multiple different treated samples for the dilution profile assays. Platelet aggregation assays are performed for each of these samples to obtain a dilution profile over the range of different concentrations. The data is obtained and measured. In this case, the platelet aggregation results are analyzed and compared against each other. If LTAAs were used as the platelet aggregation assay, the change in PA, PS, AUC, LP, DA, or FA, or a combination thereof from differing amounts of synthetic collagen are studied, and/or compared against the baseline (pre-aspirin) LTAA to determine the donor's aspirin sensitivity response (whether aspirin hypersensitive, normal/average aspirin sensitive or aspirin non-responsive and the degree of sensitivity therein). In certain embodiments the results are analyzed using the aggregometer's proprietary algorithm embedded in system software.

The inventors have determined that this embodiment can be used to predict the donor's platelet aspirin response. For individuals having an average or “normal” aspirin sensitivity, when looking at the slope, percentage aggregation and lag phase or the AUC, over the dilution profile, the slope, percentage aggregation or the AUC will show a corresponding decrease along with the decrease in the amount of synthetic collagen used. Thus, for example, within a given range of various dilutions of synthetic collagen, as the concentration goes down, so will the slope, percentage aggregation, and the AUC. There seems to be an almost linear decrease in slope, percentage aggregation and AUC that runs almost parallel or has almost a direct correlation with the concentration of synthetic collagen. On the other hand, for individuals who are aspirin non-responders, instead of having a slope, percentage aggregation, and AUC that has a linear-like decrease that corresponds with the decrease in synthetic collagen, there is a point along the dilution profile where there is an increase in slope, an unexpected temporary increase in percentage aggregation and/or a temporary increase in AUC when there should be a decrease (because the concentration of synthetic collagen decreases). Then, as the dilution profile continues to decrease, the slope and AUC “bounce back” down to where it should be (based on the dilution of synthetic collagen) and where it was before the temporary increase and then continues along decreasing. The point at which the bounce back will occur will be at different concentrations of synthetic collagen, and will depend upon the aspirin dose, as well as the patient's metabolic and platelet receptor genetics.

Aspirin hypersensitive individuals will show increases in PA, PS and AUC compared to expected/normal results.

In certain embodiments of the invention utilizing the dilution profile LTAA concept, a series of 7, 6 or 5 different concentrations are used to develop a dilution profile, and in other embodiments, 4 different concentrations are used and yet in other embodiments, 3 or 2 different concentrations are used. Dilution profiles, when presented graphically have distinctive shapes that may be further visually assessed rapidly much like an EKG. Using too many different concentrations can make the test cumbersome and time consuming, whereas using too few concentrations reduces the amount of data obtained and limits the sensitivity analysis.

The range of synthetic collagen used is preferably within the “aspirin sensitive range,” which is defined herein as the range of concentrations in which in an average aspirin sensitive individual the measured platelet activity/aggregation is reduced corresponding with decreasing amounts of synthetic collagen concentrations (e.g. the AUC and/or the slope decreases with the concentration of collagen).

In certain embodiments the different synthetic collagen dilution amounts comprise multiple different synthetic collagen amounts chosen from within a concentration range that is low enough to discount the effects of the anti-platelet medication but still pick up the effects of the aspirin. In certain embodiments, there are different synthetic collagen amounts (2, 3, 4, 5, 6 or 7 different dilutions) each chosen from with the range of 0.01 ng/mL to 1.0 ng/mL; or chosen from with the range of 0.1 ng/mL to 0.5 ng/mL; or chosen from with the range of 0.1 ng/mL to 1.0 ng/mL; or chosen from with the range of 0.1 ng/mL to 1.5 ng/mL; or chosen from with the range of 0.5 ng/mL to 2.0 ng/mL. As some non-limiting examples, some embodiments have 5 different dilutions chosen from within the range of 0.01 ng/mL to 1.0 ng/mL such as 0.01 ng/mL, 0.05 ng/mL, 0.1 ng/mL, 0.5 ng/mL, and 1.0 ng/mL. As some non-limiting examples, some embodiments have 6 different dilutions chosen from within the range of 0.5 ng/mL to 2.0 ng/mL such as 0.5 ng/mL, 0.75 ng/mL, 1.0 ng/mL, 1.25 ng/mL, 1.75 ng/mL and 2.0 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

E. Testing for Residual Platelet Activity in an Individual on Dual Anti-Platelet Medication Therapy (how Active are the Platelets after Exposure to Anti-Platelet Medication and Aspirin).

One embodiment of the present invention provides tests that can determine the individual's residual platelet activity to the dual therapy (aspirin and an anti-platelet medication) when the individual is on a dual anti-platelet medication therapy (i.e. on aspirin and an anti-platelet medication). In other words, in this embodiment, a level of synthetic collagen is used that is sensitive to both aspirin and the anti-platelet medication. This test informs the physician the overall platelet sensitivity/reactivity in the individual. This test is very useful in the emergency setting when the physician primarily just needs to know the patients platelet status and doesn't necessarily need to know the individual effect of the aspirin or the anti-platelet medication on the platelet reactivity. Typically, anti-platelet medications have been broken down into 5 classes of anti-platelet drugs based on their mechanism of action. The chart below provides some examples. Note that although aspirin is considered an anti-platelet medication, in the present application when a dual therapy is referred to, it is meant as a therapy including aspirin and a second anti-platelet medication (that is not aspirin).

| Class | Example | Therapeutic Basis |
|---|---|---|
| 1 | Aspirin | Salicylate (COX Inhibitor) |
| | | IUPAC Name: 2-acetyloxybenzoic acid |
| 2 | Dipyridimole | Phosphodiesterase Inhibitor |
| | | (cAMP and cAMP-inhibited cGMP 3′,5′- |
| | | cyclic phosphodiesterase 10A Activity |
| | | Inhibitor) |
| | | IUPAC Name: 2-[[2- |
| | | [bis(2-hydroxyethyl)amino]- |
| | | 4,8-di(piperidin-1-yl)pyrimido[5,4- |
| | | d]pyrimidin-6-yl]- |
| | | (2-hydroxyethyl)amino]ethanol |
| 3 | Reopro ® | Not listed as Pubchem Compound |
| | | Immunoglobulin Fragment Fab Fragment |
| | | Chimeric monoclonal antibody 7E3 |
| | | (GP IIbIIIa receptor blocker) |
| 4 | Plavix ® | Thienopiryridine (PY2 receptor inhibitor) |
| | | IUPAC Name: methyl (2S)-2-(2- |
| | | chlorophenyl)-2-(6,7-dihydro-4H- |
| | | thieno[3,2-c]pyridin-5-yl)acetate |
| 5 | Brilinta ® | Cyclopentyltriazolopyrimidine (P2Y₁₂ Inhibitor) |
| | | IUPAC Name: (1S,2S,3R,5S)- |
| | | 3-[7-[[(1R,2S)-2-(3,4- |
| | | difluorophenyl)cyclopropyl]amino]- |
| | | 5-propylsulfanyltriazolo[4,5- |
| | | d]pyrimidin-3-yl]-5- |
| | | (2-hydroxyethoxy)cyclopentane-1,2-diol |
| 6 | Aggregnox ® | Phosphodiesterase Inhibitor |
| | | (cAMP-specific 3′,5′-cyclic |
| | | phosphodiesterase 4A Inhibitor) |
| | | IUPAC Name: |
| | | 2-[[2-[bis(2-hydroxyethyl)amino]- |
| | | 4,8-di(piperidin-1- |
| | | yl)pyrimido[5,4-d]pyrimidin-6-yl]- |
| | | (2-hydroxyethyl)amino]ethanol |

Note:
some individuals consider Brilanta ® as a Class 4 drug because of chemical similarities

This method of the present invention involves performing one platelet aggregation study, preferably a LTAA, whereby a platelet rich sample is obtained from an individual and is combined with synthetic collagen to form a treated sample. The sample is placed into a LTA aggregometer and the light transmission through the treated sample is obtained to get a readout to determine the individual's percent aggregation. In certain embodiments, an initial LTA may be performed to check for spontaneous aggregation to test for whether the platelets have any inherent hyperactivity. Then, depending upon the levels of aggregation or (as read through PA, PS, AUC, LP, DA, or FA), if the individual shows a significant reduction in platelet aggregation, then the individual may be characterized as normal or having an average platelet reactivity/sensitivity. If the individual shows very little inhibition of platelet aggregation (shows high platelet activity) (i.e. the platelets still aggregated after the individual ingested the dual anti-platelet medication), then the individual may be characterized as being non-responsive. If the individual showed an almost complete lack of platelet aggregation after ingesting the dual anti-platelet medication, then the individual may be characterized as being hypersensitive. The readout from the LTA may be slope, primary aggregation, area under the curve, lag phase, disaggregation (DA) or final aggregation (FA), or a combination thereof. In certain embodiments, an algorithm that combines and categorizes this data, into an actionable form useful to the physician may be employed.

In other words, this tests the residual platelet activity—how reactive are the platelets (likely to aggregate) after the patient has ingested the dual therapy of an anti-platelet medication and aspirin. In these embodiments, the concentration of synthetic collagen is such that the effect of both the anti-platelet medication and the aspirin on platelet aggregation is taken into account and the activity of the platelets is the activity that remains after the medications have had their effect. Preferably in these embodiments the final in-test concentration of synthetic collagen used preferably ranges from about 25 ng/mL to 35 ng/mL; or is about 2.0 ng/mL; or ranges from 2.0 ng/mL to 12.5 ng/mL; or ranges from about 2.0 ng/mL to about 25 ng/mL; or ranges from about 2.0 ng/mL to about 35 ng/mL; or ranges from about 2.0 ng/mL to about 39 ng/mL; or is about 12.5 ng/mL; or ranges from about 12.5 ng/mL to about 25 ng/mL; or ranges from about 12.5 ng/mL to about 35 ng/mL; or ranges from about 12.5 ng/mL to about 39.0 ng/mL; or ranges from about 25 ng/mL to about 39 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

### F. Testing for Residual Platelet Activity in Patient on Dual Therapy Using a Dilution Profile

In another embodiment for testing residual platelet activity in an individual on a dual anti-platelet medication therapy, a dilution profile of synthetic collagen is utilized across a number of different platelet aggregation assays, such as LTAAs or flow cytometery run on an individual's PRP or whole blood sample. In this embodiment, a baseline aggregation assay is performed before the patient begins the dual therapy. The baseline aggregation assay may be one test or may be a dilution profile baseline. The baseline results of the individual test or dilution profile are compared against the dilution profile results obtained after the patient has been on the dual therapy. In this embodiment, multiple different PRP or whole blood samples are obtained from the individual and each platelet sample is mixed with a different amount of synthetic collagen and a platelet aggregation assay, such as LTAA or flow cytometry, is performed on each sample to obtain a dilution profile. Preferably the same type of platelet aggregation assay is used throughout the test. For example, LTAAs are used to measure platelet aggregation for each sample. Also, if a dilution profile is used for the baseline, preferably the same dilutions are used to test the platelets after the patient has been on the dual therapy.

The results are analyzed and the change in platelet aggregation as seen in changes in the PA, PS, AUC, LP, DA, or FA, from the baseline compared to the tests after the patient has been on the dual therapy, as well as a comparison of aggregation over the differing amounts of synthetic collagen, are studied (often using an algorithm that categorizes the data or information and reports the data) to determine the individual's residual platelet reactivity. In certain embodiments the results are characterized using the aggregometer's proprietary algorithm embedded in system software, which makes the results of the analysis easier for the diagnostician to understand and make appropriate clinical decisions.

In other embodiments, a pre-dual therapy baseline or baseline profile is not obtained. This may be useful in the emergency clinical setting when it is not feasible to obtain a pre-dual therapy medication baseline or whether one cannot determine from the patient whether he or she has been on a dual therapy. In this embodiment, multiple different platelet rich plasma samples or whole blood samples are obtained from the individual and each are mixed independently with a different synthetic collagen concentration to obtain multiple different treated samples for the dilution profile assays. Platelet aggregation assays are performed for each of these samples to obtain a dilution profile over the range of different concentrations. The data is obtained and measured. In this case, the platelet aggregation results are analyzed and compared against each other. If LTAAs were used as the platelet aggregation assay, the change in PA, PS, AUC, LP, DA, or FA, or a combination thereof from differing amounts of synthetic collagen are studied, to determine the donor's residual platelet activity. In certain embodiments the results are analyzed using the aggregometer's proprietary algorithm embedded in system software.

In certain embodiments of the invention utilizing the dilution profile LTAA concept, a series of 7, 6 or 5 different concentrations are used to develop a dilution profile, and in other embodiments, 4 different concentrations are used and yet in other embodiments, 3 or 2 different concentrations are used. Dilution profiles, when presented graphically have distinctive shapes that may be further visually assessed rapidly much like an EKG. Using too many different concentrations can make the test cumbersome and time consuming, whereas using too few concentrations reduces the amount of data obtained and limits the sensitivity analysis.

The range of synthetic collagen used is preferably within the “aspirin sensitive range,” which is defined herein as the range of concentrations in which in an average aspirin sensitive individual the measured platelet activity/aggregation is reduced corresponding with decreasing amounts of synthetic collagen concentrations (e.g. the AUC and/or the slope decreases with the concentration of collagen).

In these embodiments, the concentration of synthetic collagen is such that the effect of both the anti-platelet medication and the aspirin on platelet aggregation is taken into account and the activity of the platelets is the activity that remains after the medications have had their effect. In certain embodiments, there are different synthetic collagen amounts (2, 3, 4, 5, 6 or 7 different dilutions) each chosen from within the range of about 2.0 ng/mL to about 12.5 ng/mL; or from within the range of about 2.0 ng/mL to about 25 ng/mL; or from within the range of about 2.0 ng/mL to about 35 ng/mL; or from within the range of about 2.0 ng/mL to about 39 ng/mL; or from within the range of about 12.5 ng/mL to about 25 ng/mL; from within the range of about 12.5 ng/mL to about 39.0 ng/mL; or from within the range of about 25 ng/mL to about 39 ng/mL. As a non-limiting example, 5 different concentrations are used chosen from within the range of 12.5 ng/mL to about 35 ng/mL are as follows: 12.5 ng/mL, 20 ng/mL, 25 ng/mL, 30 ng/mL and 35 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

### G. Combination of Tests

Any of the tests described above can be combined for a more in-depth analysis of an individual's platelet activity in response to the dual anti-platelet medication therapy. For a particular patient, for an example, an LTAA could be run using an amount of synthetic collagen that provides the physician with an insight as to how the anti-platelet medication alone affects the platelet reactivity (e.g. about 50 ng/mL final in-test concentration of synthetic collagen)(by discounting the aspirin effects on the platelets). Then the physician could run another LTAA using an amount of synthetic collagen that provides the physician with an insight as to how the aspirin alone affects the platelet reactivity (e.g. about 1.0 ng/mL to about 0.1 ng/mL)(by discounting the anti-platelet medication effects on the platelets). Then finally, the physician could run another LTAA using an amount of synthetic collagen that provides the physician with an insight as to how the combination of the aspirin and the anti-platelet medication affects the platelet reactivity (e.g. about 25 ng/mL to 35 ng/mL), and thus provides information about the residual platelet activity. In any of these tests or combinations, a baseline may be obtained prior to ingesting the medication or aspirin, and further the tests may be run using the dilution profile concept described herein above.

### H. Compliance Testing

The present invention also provides tests that can be used to check the patient for compliance with the aspirin, anti-platelet medication and/or the dual therapy. As mentioned above, compliance means both taking the medication and taking the medicine at the right time (according to the prescribed dosing schedule). For example, the patient may be complying with the anti-platelet medication but may not be complying with the aspirin therapy or vice versa. The present invention provides a mechanism to test the patient for his compliance. Non-compliance includes not taking the medication, not taking the proper dose or not staying with the effective dosing (time) schedule. Recent studies have shown that a large problem in health care is patient noncompliance with aspirin and other therapies. Current thinking is that what was once thought to be aspirin resistance may instead be a manifestation of non-compliance complicated by the use of multiple, non-standardized laboratory tests to evaluate platelets inhibited response to aspirin.

### I. Compliance of Aspirin Therapy Regimen

Accordingly, to measure compliance the patient can be routinely tested, such as once a week, bi-monthly, monthly, every 3 months, etc., and the results compared against each other. If the aggregation results vary widely from one test to another, the patient can be further tested to determine if aspirin resistance has developed or the patient could be questioned as to his compliance in taking the prescribed doses of aspirin. If it is suspected that the patient has not been taking the aspirin or not taking it within the dosing window, the patient's plasma can be treated with aspirin and then tested. If aggregation appears in the aspirinated sample, then it may be concluded that the patient had not been taking the aspirin as directed. In some cases, the patient may be taking the aspirin sporadically and not at the same time each day. The aggregation tests may reveal variability from test to test and this variability could be used as an indicator that the patient has not been following the prescribed regular dosing regimen (either not taking the dose every day or taking the dose at different times of the day). It has been found that a patient on aspirin therapy that does not comply with the therapy but not taking the aspirin every day or taking it at different times of the day actually puts the patient at a higher than baseline levels for risk of a thrombotic event. If aggregation does not appear in the aspirinated sample, it could be that the patient had developed aspirin resistance. Further testing could be performed to determine if the patient should be on a different dual therapy of two different anti-platelet medication or perhaps a regimen a different anti-platelet medication altogether without aspirin.

In these tests, the concentration of synthetic collagen would be is low enough to be insensitive to the effects of the anti-platelet medication on platelet activity but high enough to be sensitive to the effects of aspirin on platelet activity. In other words, in these embodiments the final in-test concentration of synthetic collagen that is used tests the ability of the platelets to aggregate in the presence of an agonist (synthetic collagen) and is at a concentration where the test ignores the effect of the anti-platelet medication on platelet aggregation but still measures the effect of the aspirin on the platelet aggregation. The concentration of synthetic collagen in this is aspect is a low range and is actually so low that a biological collagen could not be diluted to this low concentration. Preferably, in these embodiments the final in-test concentration of synthetic collagen used ranges from about 0.01 ng/mL to about 1.0 ng/mL; or ranges from about 0.1 ng/mL to about 0.5 ng/mL; or ranges from about 0.1 ng/mL to about 1.0 ng/mL; or ranges from about 0.1 ng/mL to about 1.5 ng/mL; or is from about 0.5 ng/mL or less; or ranges from about 0.5 ng/mL to about 2.0 ng/mL; or is less than 2.0 ng/mL; or is less than 10 ng/mL; or is less than 5 ng/mL. These values and ranges are preferably used when the platelet aggregation tests are light transmission assays and the sample is a PRP sample that is tested.

### J. Compliance of Anti-Platelet Medication Therapy Regimen

If the aggregation results vary widely from one test to another, the patient can be further tested to determine if resistance has developed to the anti-platelet medication or the patient could be questioned as to his compliance in taking the prescribed doses of the anti-platelet medication. If it is suspected that the patient has not been taking the anti-platelet medication or not taking it within the dosing window, the patient's plasma can be treated with the anti-platelet medication and then tested. If aggregation appears in the treated sample, then it may be concluded that the patient had not been taking the anti-platelet medication as directed. In some cases, the patient may be taking the aspirin sporadically and not at the same time each day. If aggregation does not appear in the treated sample, it could be that the patient had developed resistance to the anti-platelet medication. Further testing could be performed to determine if the patient should be on a different dual therapy of two different anti-platelet medication or perhaps a regimen a different anti-platelet medication altogether without aspirin.

In these embodiments the final in-test concentration of synthetic collagen that is used tests the ability of the platelets to aggregate in the presence of an agonist (synthetic collagen) and is at a concentration where the test ignores the effect of aspirin on platelet aggregation but still measures the effect of the anti-platelet medication on the platelet aggregation. Preferably, in these embodiments the final in-test concentration of synthetic collagen used ranges from about 50 ng/mL to about 500 ng/mL; or is >40 ng/mL; or is >50 ng/mL; or ranges from about 40 to about 500 ng/mL; or ranges from about 40 to about 400 ng/mL; or ranges from about 40 to 300 ng/mL; or ranges from about 40 to about 200 ng/mL; or ranges from about 40 to about 100 ng/mL; or ranges from about 40 to about 90 ng/mL; or ranges from about 40 to about 80 ng/mL; or ranges from about 40 to about 70 ng/mL; or ranges from about 40 to about 60 ng/mL; or ranges from about 50 to about 400 ng/mL; or ranges from about 50 to about 300 ng/mL; or ranges from about 50 to about 200 ng/mL; or ranges from about 50 to about 100 ng/mL. These values and ranges are preferably used when the platelet aggregation tests are light transmission assays and the sample is a PRP sample that is tested.

### K. Compliance of the Dual Therapy

If the aggregation results vary widely from one test to another, the patient can be further tested to determine if resistance has developed to the dual therapy or the patient could be questioned as to his compliance in taking the prescribed doses of the dual therapy. If it is suspected that the patient has not been taking the dual therapy or not taking it within the prescribed dosing window, the patient's plasma can be treated with the anti-platelet medication and then tested and can also be treated with aspirin and then tested. If aggregation appears in the treated sample, then it may be concluded that the patient had not been taking the medication(s) as directed. In some cases, the patient may be taking the aspirin and/or anti-platelet medication sporadically and not at the same time each day. If aggregation does not appear in the treated sample, it could be that the patient had developed resistance to the dual therapy. Further testing could be performed to determine if the patient should be on a different dual therapy of two different anti-platelet medication or perhaps a regimen a different anti-platelet medication altogether without aspirin.

In these embodiments, the concentration of synthetic collagen is such that the effect of both the anti-platelet medication and the aspirin on platelet aggregation is taken into account and the activity of the platelets is the activity that remains after the medications have had their effect. Preferably in these embodiments the final in-test concentration of synthetic collagen used preferably ranges from about 25 ng/mL to 35 ng/mL; or is about 2.0 ng/mL; or ranges from 2.0 ng/mL to 12.5 ng/mL; or ranges from about 2.0 ng/mL to about 25 ng/mL; or ranges from about 2.0 ng/mL to about 35 ng/mL; or ranges from about 2.0 ng/mL to about 39 ng/mL; or is about 12.5 ng/mL; or ranges from about 12.5 ng/mL to about 25 ng/mL; or ranges from about 12.5 ng/mL to about 35 ng/mL; or ranges from about 12.5 ng/mL to about 39.0 ng/mL; or ranges from about 25 ng/mL to about 39 ng/mL. These values and ranges are preferably used when the platelet aggregation tests are light transmission assays and the sample is a PRP sample that is tested.

In the methods/assays of the present invention, instead of having the patient ingest aspirin and thereafter take a blood sample, a sample of blood can be taken before any aspirin is ingested and the sample can be “aspirinated” (or “aspirinized”)(that is, an aspirin solution (may also be the lysine salt of aspirin or Aspisol®) is added to the PRP and then tested). It has been determined that in the all of the methods/assays of the present, either the patient can ingest the aspirin or the sample can be aspirinated. This can speed up the testing because the patient does not need to ingest the aspirin and have time pass to allow the aspirin to get into the patient's system. Instead, the blood is drawn and a PRP sample is obtained, and one part is aspirinated and the other part is not, thus also allowing the two samples to be tested side by side.

In some cases, the aspirinized portion of the test can be manufactured by adding aspirin to the PRP to a desired final concentration. In certain embodiments, the concentration of the aspirin ranges from 25 to 150 μM, 25-100 μM, 50-150 μM, 50-100 μM, 75-150 μM, 75-100 μM and preferably a final concentration of 100 μM.

### L. Method of Predicting Patient's Response to Aspirin, an Anti-Platelet Medication or Dual Therapy

The present invention also provides a method of determining or calculating whether a patient would benefit from a certain prescription of aspirin, anti-platelet or dual therapy as well as what class of anti-platelet drug is best suited for the patient (personalized medicine/therapy).

### L1. Predicting Effectiveness of Aspirin Therapy

For example, if a physician was contemplating starting a patient on aspirin therapy, the patient's PRP sample (or whole blood sample) can be aspirinated (or aspirinized) and then can be tested for platelet aggregation using means known in the art, such as LTAAs. If the resulting platelet aggregation tests showed that the platelets did not aggregate to a desired healthy level after being treated with aspirin, then the physician may not want prescribe an aspirin therapy since the patient would seem to be insensitive to aspirin. In other words, the physician could use the results of the LTAAs to predict whether aspirin therapy would be beneficial or detrimental to the patient based on the amount of platelet aggregation that occurred in the presence of the synthetic collagen and the aspirin. In some instances, it may be that the patient's platelets aggregated too strongly and contained no residual activity. In this case, the physician may not prescribe an aspirin therapy regimen as this patient may be susceptible to bleeding complications. In this case the physician would predict that the aspirin was too effective in inhibiting platelet aggregation, would choose another anti-platelet drug.

In this embodiment, the final in-test concentration of synthetic collagen used ranges from about 0.01 ng/mL to about 1.0 ng/mL; or ranges from about 0.1 ng/mL to about 0.5 ng/mL; or ranges from about 0.1 ng/mL to about 1.0 ng/mL; or ranges from about 0.1 ng/mL to about 1.5 ng/mL; or is from about 0.5 ng/mL or less; or ranges from about 0.5 ng/mL to about 2.0 ng/mL; or is less than 2.0 ng/mL; or is less than 10 ng/mL; or is less than 5 ng/mL. These values and ranges are preferably used when the platelet aggregation tests are light transmission assays and the sample is a PRP sample that is tested.

### L2. Predicting Effectiveness of Dual-Therapy Regimen

In addition, since the inventors have developed tests that can discount the effect of an anti-platelet medication on platelet aggregation by using a certain concentration range of synthetic collagen in the platelet aggregation assays, tests of the present invention can be used on patients who are currently on an anti-platelet medication and whom the physician might be considering supplementing with an aspirin therapy regimen. Thus, the present invention provides a method of predicting the effectiveness of a dual therapy regimen (predicting the effect of adding aspirin to a patient's anti-platelet medication therapy regimen). In this situation, a whole blood sample or PRP sample is taken from the patient while he is on the anti-platelet medication. The samples are then aspirinized and tested for platelet aggregation. If the resulting platelet aggregation tests showed that the platelets did not aggregate to a desired healthy level after being treated with aspirin, then the physician may not prescribe an aspirin therapy since the patient would seem to be insensitive to aspirin (the tests predicts that the aspirin therapy would not be effective). If the patient's platelets aggregated too strongly and contained no residual activity, the physician may not prescribe an aspirin therapy regimen as this patient may be susceptible to bleeding complications (the tests predicts that the aspirin therapy would be too effective). If the platelets showed an acceptable level of aggregation, then the physician may consider prescribing the dual therapy of the anti-platelet medication and the aspirin therapy (the tests predict a desired or acceptable level of platelet aggregation).

In this embodiment, the concentration of synthetic collagen is low enough to be insensitive to the effects of the anti-platelet medication on platelet activity but high enough to be sensitive to the effects of aspirin on platelet activity. In other words, in these embodiments the final in-test concentration of synthetic collagen that is used tests the ability of the platelets to aggregate in the presence of an agonist (synthetic collagen) and is at a concentration where the test ignores the effect of the anti-platelet medication on platelet aggregation but still measures the effect of the aspirin on the platelet aggregation. Preferably, in these embodiments the final in-test concentration of synthetic collagen used ranges from about 0.01 ng/mL to about 1.0 ng/mL; or ranges from about 0.1 ng/mL to about 0.5 ng/mL; or ranges from about 0.1 ng/mL to about 1.0 ng/mL; or ranges from about 0.1 ng/mL to about 1.5 ng/mL; or is from about 0.5 ng/mL or less; or ranges from about 0.5 ng/mL to about 2.0 ng/mL; or is less than 2.0 ng/mL; or is less than 10 ng/mL; or is less than 5 ng/mL. These values and ranges are preferably used when the platelet aggregation tests are light transmission assays and the sample is a PRP sample that is tested.

L3. Predicting Effectiveness of Anti-Platelet Medication (when Patient is on Aspirin Therapy)

Further, the present invention also provides assays that discount the effect of aspirin on platelet aggregation and can test the effect of an anti-platelet medication on the platelet activity. Thus, if a patient were on an aspirin therapy and the physician was considering starting the patient on a dual therapy by adding an anti-platelet medication, the patient's PRP or whole blood sample could be taken and treated with an anti-platelet medication. After adding synthetic collagen, platelet aggregation is studied. If it is determined that the level of platelet aggregation is acceptable, then the physician may prescribe that medication. Or if the level of platelet aggregation was not acceptable, the physician may test and prescribe a different anti-platelet medication.

In these embodiments the final in-test concentration of synthetic collagen that is used tests the ability of the platelets to aggregate in the presence of an agonist (synthetic collagen) and is at a concentration where the test ignores the effect of aspirin on platelet aggregation but still measures the effect of the anti-platelet medication on the platelet aggregation. Preferably, in these embodiments the final in-test concentration of synthetic collagen used ranges from about 50 ng/mL to about 500 ng/mL; or is >40 ng/mL; or is >50 ng/mL; or ranges from about 40 to about 500 ng/mL; or ranges from about 40 to about 400 ng/mL; or ranges from about 40 to 300 ng/mL; or ranges from about 40 to about 200 ng/mL; or ranges from about 40 to about 100 ng/mL; or ranges from about 40 to about 90 ng/mL; or ranges from about 40 to about 80 ng/mL; or ranges from about 40 to about 70 ng/mL; or ranges from about 40 to about 60 ng/mL; or ranges from about 50 to about 400 ng/mL; or ranges from about 50 to about 300 ng/mL; or ranges from about 50 to about 200 ng/mL; or ranges from about 50 to about 100 ng/mL. These values and ranges are preferably used when the platelet aggregation tests are light transmission assays and the sample is a PRP sample that is tested.

### L4. Predicting Effectiveness of Anti-Platelet Therapy

Further, the present invention also provides assays that predict the effectiveness of an anti-platelet medication on platelet activity. See example 2. Thus, if a physician was considering starting a patient on an anti-platelet medication, the patient's PRP or whole blood sample could be taken and treated with an anti-platelet medication. After adding synthetic collagen, platelet aggregation is studied with or without aspirin in the sample. If it is determined that the level of platelet aggregation is acceptable, then the physician may prescribe that medication. Or if the level of platelet aggregation was not acceptable, the physician may test and prescribe a different anti-platelet medication.

In these embodiments the final in-test concentration of synthetic collagen that is used tests the ability of the platelets to aggregate in the presence of an agonist (synthetic collagen) Preferably, in these embodiments the final in-test concentration of synthetic collagen used ranges from about 12.5 ng/mL to about 100 ng/mL; or from about 50 ng/mL to about 500 ng/mL; or is >40 ng/mL; or is >50 ng/mL; or ranges from about 40 to about 500 ng/mL; or ranges from about 40 to about 400 ng/mL; or ranges from about 40 to 300 ng/mL; or ranges from about 40 to about 200 ng/mL; or ranges from about 40 to about 100 ng/mL; or ranges from about 40 to about 90 ng/mL; or ranges from about 40 to about 80 ng/mL; or ranges from about 40 to about 70 ng/mL; or ranges from about 40 to about 60 ng/mL; or ranges from about 50 to about 400 ng/mL; or ranges from about 50 to about 300 ng/mL; or ranges from about 50 to about 200 ng/mL; or ranges from about 50 to about 100 ng/mL. These values and ranges are preferably used when the platelet aggregation tests are light transmission assays and the sample is a PRP sample that is tested.

The concentration of medicine that is used in the test depends on the medication and the concentration of the synthetic collagen. Because synthetic collagen induced platelet aggregation is a functional test, there is no need to have genetic and metabolic test data available prior to prescribing the individualized anti-platelet therapy. The concentration of medicine that is used may depend on the dosing that is given to a patient and the desired levels obtained in the plasma. To determine the best concentrations to use in the method of the invention, one skilled in the art could follow example 2 and obtain healthy donors and test varying ranges of the anti-platelet medication and the synthetic collagen, using the plasma levels as a guide to determine the best ranges (the most sensitive and most reliable across a collection of healthy donors) of anti-platelet medication and synthetic collagen.

L5. Predicting Residual Platelet Activity when Patient is on Dual Therapy

In another embodiment, the physician may wish to test the residual platelet activity that remains after the plates have been exposed to aspirin and the anti-platelet medication as a method of predicting a patient's residual platelet activity while on a dual therapy regimen. In this case the patient may be on aspirin therapy and the sample is treated with an anti-platelet medication, or the patient may be on an anti-platelet medication and the sample is treated with aspirin or it may be that the patient may already be on a dual therapy. In these embodiments, the concentration of synthetic collagen is such that the effect of both the anti-platelet medication and the aspirin on platelet aggregation is taken into account and the activity of the platelets is the activity that remains after the medications have had their effect. Preferably in these embodiments the final in-test concentration of synthetic collagen used preferably ranges from about 25 ng/mL to 35 ng/mL; or is about 2.0 ng/mL; or ranges from 2.0 ng/mL to 12.5 ng/mL; or ranges from about 2.0 ng/mL to about 25 ng/mL; or ranges from about 2.0 ng/mL to about 35 ng/mL; or ranges from about 2.0 ng/mL to about 39 ng/mL; or is about 12.5 ng/mL; or ranges from about 12.5 ng/mL to about 25 ng/mL; or ranges from about 12.5 ng/mL to about 35 ng/mL; or ranges from about 12.5 ng/mL to about 39.0 ng/mL; or ranges from about 25 ng/mL to about 39 ng/mL. These values and ranges are preferably used when the platelet aggregation tests are light transmission assays and the sample is a PRP sample that is tested.

In any of the tests described herein, any measurement of platelet aggregation or inhibition of platelet aggregation may be used. In certain preferred embodiments, LTAAs are used. In certain other preferred embodiments, flow cytometry is used to measure platelet aggregation.

As seen from the discussion above concerning the various embodiments, it is apparent that varying amounts of synthetic collagen can be used, depending upon the test to be performed or the clinician's request for an assessment. The amount of synthetic collagen used is about two or more orders of magnitude less than what is generally used when performing LTAAs with biological source collagen. For example, usually LTAAs using calf skin biological collagen generally use 0.19 mg/mL (milligrams/mL) collagen (as the “in-test” concentration); and LTAAs using equine tendon collagen generally use 2.0 μg/mL (micrograms/mL) collagen in the LTAA test (as the “in-test” concentration), whereas generally the methods of the present invention utilize from about 500 ng/mL to about 0.10 ng/mL (nanogram/mL) of synthetic collagen in each LTAA test (as the “in-test” concentration).

When testing platelet aggregation using flow cytometry, the usual concentrations of biological collagen ranges from 0.01-100 μg/mL, with 20 μg/mL to be most common. However, using synthetic collagen, the amounts used are much lower, ranging from about 2.0 ng/mL to about 640 ng/mL.

Although there are ranges included hereinabove, the present invention is not limited by the recitation of the first and last endpoint to only mean the first and last, but expressly includes the first and last endpoint as well as all of the concentrations within the endpoints. It would be just too cumbersome herein to list every concentration that falls within the recited ranges. The inventors have contemplated using more than one concentration, and more than one range as well as more than one concentration within the recited range to produce the most sensitive and accurate results.

The ability to use such low concentrations as well as dilution profiles of collagen is only available with the synthetic collagen. When the inventors tried to dilute biological collagen down to similar low concentrations, it became physically impossible to dilute down the biological collagen to the levels anywhere close to that used in the LTAAs of the present invention with synthetic collagen. Biological collagen is an insoluble, viscous, heterogeneous material, and has long structured fibrous proteins wound into a triple helix. These physical properties precluded the ability to dilute the biological collagen to any low concentration even 100 fold close to the synthetic collagen or to have any such dilution perform in a predictable or useful manner. Further the LTAAs did not work (no aggregation occurred) when using calf skin collagen at a concentration a little lower than 0.19 mg/mL (milligrams/mL) (as the “in-test” concentration); nor when using equine tendon collagen a little lower than 2.0 μg/mL (micrograms/mL).

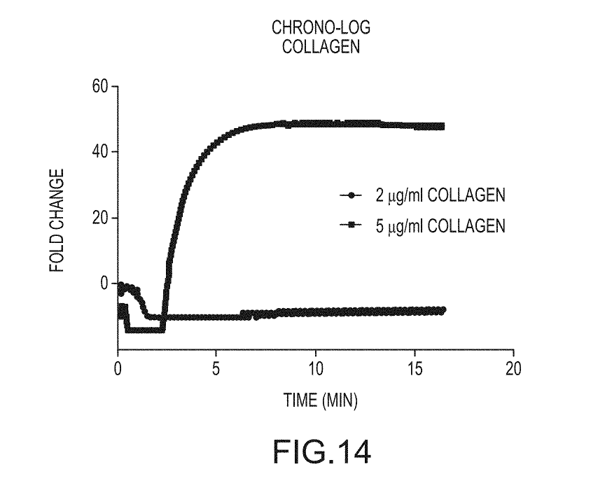

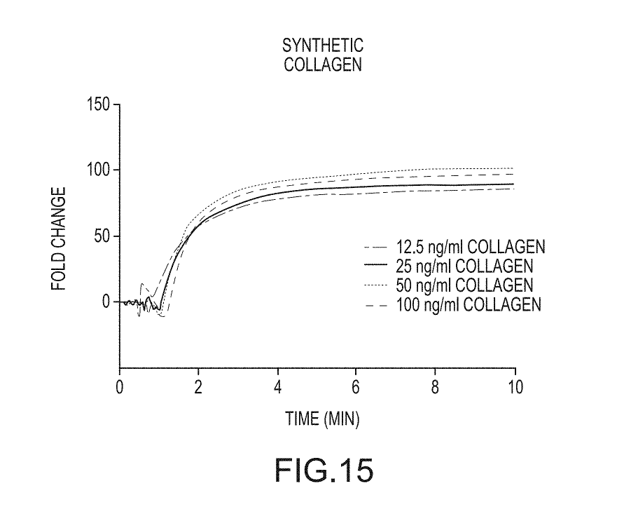

In tests performed on whole blood, using the whole blood mode of the Chrono Log aggregometer it was shown that diluting biological collagen did not produce any viable results but synthetic collagen could be diluted from 100 ng/mL to 12.5 ng/mL and still elicit the same response. See FIGS. 14 and 15.

### Synthetic Collagen

In certain embodiments the synthetic collagen is described in U.S. patent application Ser. No. 12/520,508, which is herein incorporated by reference in its entirety. In certain embodiments, the synthetic collagen is a synthetic collagen that has the ability to self-assemble into a triple helix to form fibrils, which allows the synthetic collagen to mimic type I collagen (allows the synthetic collagen to be recognized or function as type I collagen). In certain embodiments the synthetic collagen comprises a polypeptide having a peptide fragment represented by the formula (I)

-(Pro-X-Gly)ₙ (I)

wherein X represents Hyp; and n represents an integer of from 20 to 5,000; and wherein the polypeptide has a molecular weight at a range of from 10,000 to 500,000,000. In certain embodiments, the synthetic collagen having the structure of formula (I) has the ability to self-assemble into a triple helix to form fibrils, which allows the synthetic collagen to mimic type I collagen. It is preferred that synthetic collagen used in all the assays of the present invention have the ability to self-assemble into a triple helix to form fibrils, which allows the synthetic collagen to mimic human type I collagen.

In certain embodiments, the synthetic collagen that is used is described in U.S. Pat. No. 7,262,275. The synthetic collagen molecule was made by the method described in U.S. Pat. No. 7,262,275 (See e.g. Example 6 and Example 7). The molecular weight of the molecule was measured by the method described in the example section in the same patent as was over 1,000,000.

In certain embodiments the synthetic collagen has the following values based on GPC-MALs (gel permeation chromatography—multi-angle laser light scattering); Average molecular weight (Mₙ) 1.3×10⁴; M_{w} (weight average molecular weight)=1.6×10⁴; size average molecular weight (M_{z}) 2.0×10⁴. In other embodiments, the synthetic collagen as has the following values based on GPC-MALs (gel permeation chromatography—multi-angle laser light scattering); Average molecular weight (Mₙ) 2.8×10⁴; M_{w} (weight average molecular weight)=4.1×10⁴; size average molecular weight (M_{z}) 6.1×10⁴.

The synthetic collagen can be measured by GPC-Mals. The synthetic collagen molecules tested in the present invention were measured using the HLC-8120GPC device manufactured by Tosoh with the following conditions.
Column: TSKgel α-M (7.8 mm I.D.×30 cm)×2 (manufactured by Tosoh).
Density Detector: Differential refractometer (RI detector), polarity=(+).
MALS: DAWN HELEOS (manufactured by Wyatt Technology).
MALS Laser wave: 658 nm.
Eluent: HFIP (1,1,1,3,3,3-Hexfloro-2-propanol) manufactured by central glass +5 mM-CF₃COONa (1^{st} class manufactured by Wako Pure Chemical).
Flow Speed: 0.6 mL/min.
Column Temp.: 40° C.
RI detector Temp.: 40° C.
MALS Temp.: Room Temp.
Sample density: 2 mg/mL.
Sample amount: 100 μL.
Pre-treatment of sample: After weighing the samples, they were dissolved by adding a given amount of eluent and left at room temperature overnight. The samples gently mixed and then were then filtered through a 0.5 μm PTFE cartridge filter.

In certain embodiments n is an integer of 20 to 250. In certain embodiments n is an integer of 20 to 200. In certain embodiments n is an integer of 20 to 150. In certain embodiments n is an integer of 30 to 100. In certain embodiments n is an integer of 20 to 2,500; of 20 to 2,000; of 20 to 1,500; of 20 to 1,000; of 20 to 500; or of 20 to 250; 30 to 2,500; of 30 to 2,000; of 30 to 1,500; of 30 to 1,000; of 30 to 500; or of 30 to 250. It is preferred that the synthetic collagen molecules discussed above have the ability to self-assemble into a triple helix to form fibrils, which allows the synthetic collagen to mimic type I collagen.

Two factors to consider in choosing the synthetic collagen is solubility and ease of handling. If the molecular weight is too small, the synthetic collagen may have poor solubility characteristics. If the molecular weight is too large, the synthetic collagen may not have good handling characteristics (may be too viscous and may have poor dispersibility). Thus, a preferred synthetic collagen of the formula (I) [-(Pro-X-Gly)ₙ] has both good solubility and good handling characteristics.

The following synthetic collagen molecules were tested: n=24 (Mn=6,300); n=28 (Mw=7,500); n=49 (Mn=13,000); n=60 (Mw=16,000); n=75 (Mz=20,000); n=105 (Mn=28,000); n=153 (Mw=41,000); n=229 (Mz=61,000). When testing various synthetic molecules, those having the n value from between 49-75 showed the best combination of desirable solubility and handling characteristics.

In certain embodiments of the invention, the synthetic collagen may be all one length (for example where n=49 for all synthetic molecules and in certain embodiments, the synthetic collagen may be a mixture of many different lengths (for example, but not limited to, the synthetic collagen is a mixture of molecule having n from 49-75).

### Kits

The present invention also provides kits useful for testing platelet aggregation, comprising a synthetic collagen. The synthetic collagen is as described above and can be at many different concentrations. In addition, the kit may comprise one or more diluents as well as controls.

The synthetic collagen can be supplied at a higher concentration in the vial than what would be used as the “in-test” concentration. In certain embodiments, the synthetic collagen in the vial is preferably more than 10 times the amount of the final “in-test” concentration desired.

In certain embodiments the synthetic collagen is supplied in the kit at the concentration contemplated for use in the methods of the present invention to bypass the need to create dilutions of the synthetic collagen. In other words, the synthetic collagen is provided so that it is in the concentration that would be used directly in the methods of the present inventions.

In other embodiments the vial could contain a higher concentration amount and the directions included in the kit would provide instructions on the desired concentration to use in the assay to achieve the desired final “in-test” concentration of synthetic collagen.

In other embodiments, the kit contains at least one single use vial and/or at least one multiple use vial of synthetic collagen. For a single use vial, the vial would contain only the amount of synthetic collagen needed for one test. For a multiple use vial, the synthetic collagen may be supplied at the desired in-test concentration, but the vial contains more than the amount of volume needed for more than one test.

The kits of the present invention preferably contain instructions for use of the synthetic collagen in the light transmission assay using methods described herein.

In other embodiments, kits of the present invention contain more than one vial of synthetic collagen at the same concentration or in other embodiments, the kits contain more than one vial at a different concentration. Kits having more than one vial at different concentrations would be useful in the dilution profile tests of the present invention. For example, one kit of the present invention may contain vials having 7, 6, 5, 4, or 3 different concentrations of synthetic collagen ranging. Each vial would, in certain embodiments, provide the synthetic collagen at the desired final “in-test” concentration and could be supplied as a single use or a multiple use vial.

The present invention is based on measuring platelet aggregation capacity along with dilution profiles generated (in certain embodiments) using synthetic collagen and subsequent data analysis and actionable report. Other methods for measuring platelet response and other measurements to one or more anti-platelet and aspirin therapy regimens using the synthetic collagen are also expected to be useful, such as, for example, the whole blood and point of care platelet function analyzers, methods, and technologies such as impedance and multiple electrode impedance aggregometry, high shear stress, cone and plate, flow cytometry, and other-point-of care technologies and assays of platelet function or reactivity.

The inventors have discovered that the nature of the vial used to store synthetic collagen can affect the collagen by activating the collagen to some unpredictable degree. It is preferable that the container used to store the synthetic collagen does not activate the collagen to ensure that when the synthetic collagen is removed from the vial and is introduced into a test system, the degree of activation and adherence of the synthetic collagen is predictable and due only to that test system. In other words, artifacts caused by unintentional activation by the interaction of the collagen with the container are not introduced into the tests. Collagens, including synthetic collagen, stored in generic polypropylene vials or other containers are activated to an unknown degree, subsequently adhere to the container, and are thus not available to participate in the test system. The amount of collagen unavailable to the test system because it has adhered to the container and/or cap is unknown and, based on stability data, is variable. The inventors have discovered that the use of synthetic collagen that has been prepared and stored in a homopolymeric container eliminates a significant degree of variability in test results. Accordingly, it is preferred that the synthetic collagen is prepared and stored in a homopolymeric container.

Most containers that are noted as polypropylene are not a single plastic but rather are a family of plastics whose performance can be modified by including various additives during the manufacturing process. Thus, the manufacturing process itself could produce different variations of polypropylene. Further, the nature of the additives is largely unknown or disclosed to the purchaser/public as this information is considered proprietary by the manufacturers. In addition, mold release agents add another variable that could not be assessed.

The inventors discovered that containers that have the best long term stability and do not interact with the synthetic collagen have the following characteristics: a) the chemical structure is based on a specific, identical monomer that is repeated (a homopolymer—a polypropylene polymer consisting of identical monomer units); b) caps are made of the same material as the tubes; and c) the caps have an additional internal seal such as a silicone O ring or washer or have a secondary seal molded therein. Exemplary vials include cryovials and caps obtained from Simport (T310 Series); Lake Charles Manufacturing (54A series), and BD Falcon tubes 352096 series).

In addition, the inventors discovered that better stability was achieved when the synthetic collagen was diluted with physiologic saline instead of purified water.

In certain embodiments (including the methods described herein and the kits), the synthetic collagen is supplied and/or stored in a polypropylene homomer. In certain embodiments, the cap is the same material as the vial/tube. In certain embodiments, the container has an additional internal seal or a cap having a secondary seal molded therein. In certain embodiments, the container contains all of the above described characteristics.

### EXAMPLES

### Example 1: Evaluate the Use of Synthetic Collagen to Detect the Antiplatelet Activity of Ticagrelor, Cilostazol and Abciximab in Normal and Aspirinized Human Platelet Rich Plasma

### Materials: Antiplatelet Drugs

Ticagrelor (Brilinta®, Astra-Zeneca, London, UK; lot AL0153, expiration February 2014) was obtained as 90 mg tablets from the Loyola University Health System inpatient pharmacy. Tablets were ground using a mortar and pestle and subsequently dissolved in DMSO at a concentration of 10 mg/mL. The stock solution was diluted in deionized water to make working solutions of 0.5, 0.1 and 0.05 mg/mL.

Cilostazol (Pletal®, Otsuka Laboratories, Tokushima, Japan; lot 0B91M) was obtained as a powder. Cilostazol was dissolved in DMSO to make a stock solution of 5 mM. The stock solution was diluted in deionized water to make working solutions of 250, 125 and 50 μM.

Abciximab (ReoPro®, Eli-Lilly, Indianapolis, Ind.; lot 12D09AA, expiration May 2015) was obtained as a 2 mg/mL solution which was diluted in physiologic saline to make working solutions of 12.5, 25 and 50 μg/mL.

Aspirin powder was dissolved in 100% methanol to make a stock solution of 100 mM. The stock solution was diluted with deionized water to make a 1 mM working solution.

### Materials: Platelet Agonists

ADP was obtained from Bio/Data Corporation, Horsham, Pa. Each vial was reconstituted with 1 mL of deionized water to make a 100 μM working solution. The final concentration of ADP in the aggregation cuvette was 10 μM.

Arachidonic acid was obtained from Bio/Data Corporation, Horsham, Pa. Each vial was reconstituted with 0.5 mL deionized water to make a 5 mg/mL working solution. The final concentration of arachidonic acid in the aggregation cuvette was 500 μg/mL.

Biological Collagen was obtained from Bio/Data Corporation, Horsham, Pa. Each vial was reconstituted with 0.5 mL deionized water to make a 1.9 mg/mL working solution. The final concentration of Bio/Data collagen in the aggregation cuvette was 190 μg/mL.

Biological Collagen was obtained from Chrono Log Corporation, Havertown, Pa. as a 1 mg/mL solution. A working solution of 100 μg/mL was made by dilution with physiologic saline. The final concentration of Chrono Log collagen in the aggregation cuvette was 10 μg/mL.

Synthetic collagen was provided by JNC Corporation, Yokohama, Japan at working concentrations of 80, 160, 320 and 640 ng/mL. The final concentrations of synthetic collagen in the aggregation cuvette were 64, 32, 16 and 8 ng/mL.

### Methods: Blood Collection

Approval for the collection of whole blood from healthy human volunteers was granted from the Institutional Review Board of the Health Sciences Division of Loyola University Chicago. Whole blood was drawn using a double syringe technique from the antecubital vein and anticoagulated by the addition of 1 part 3.2% sodium citrate to (9 parts blood to 1 part citrate). Citrated blood was centrifuged at 80×g at room temperature for 15 minutes to make platelet rich plasma (PRP). Supernatant PRP was collected and kept at room temperature in capped tubes. The remaining citrated blood was recentrifuged at 1,100×g for 15 minutes to make platelet poor plasma (PPP). The platelet count of the PRP was determined using an ICHOR II Analyzer, Helena Laboratories, Beaumont, Tex. Platelet count in the PRP was adjusted to 250,000-300,000/μl by the addition of homologous PPP.

Six blood donors were used for this study. Each was drawn on separate days for the ‘non-aspirinized’ and ‘aspirinized’ portions of the protocol. For the ‘non-aspirinized’ portion of the protocol, one donor (CS) was excluded from the final analysis as the aggregation responses were markedly different from that of the other 5 donors. For the ‘aspirinized’ portion of the protocol, three donors had taken aspirin orally <48 hours prior to blood draw. For the other three donors, aspirin was supplemented to the PRP in vitro at a final concentration of 100 μM.

### Methods: Platelet Aggregation

Platelet aggregation was measured using a PAP 8E platelet aggregometer (Bio/Data). Each well was blanked using PPP. 25 μl of saline or antiplatelet drug and 200 μl of PRP were added to cuvettes containing magnetic stir bars and incubated for three minutes to equilibrate the sample to 37° C. 25 μl of agonist was added to each cuvette and the aggregation profile was monitored until a plateau was achieved. Results were tabulated in terms of maximal aggregation level. Under some reaction conditions, reversible aggregation was observed. This was most commonly observed with ADP and arachidonic acid-induced aggregation in the presence of ticagrelor or cilostazol. Final aggregation levels were also tabulated.

### Results:

### Ticagrelor

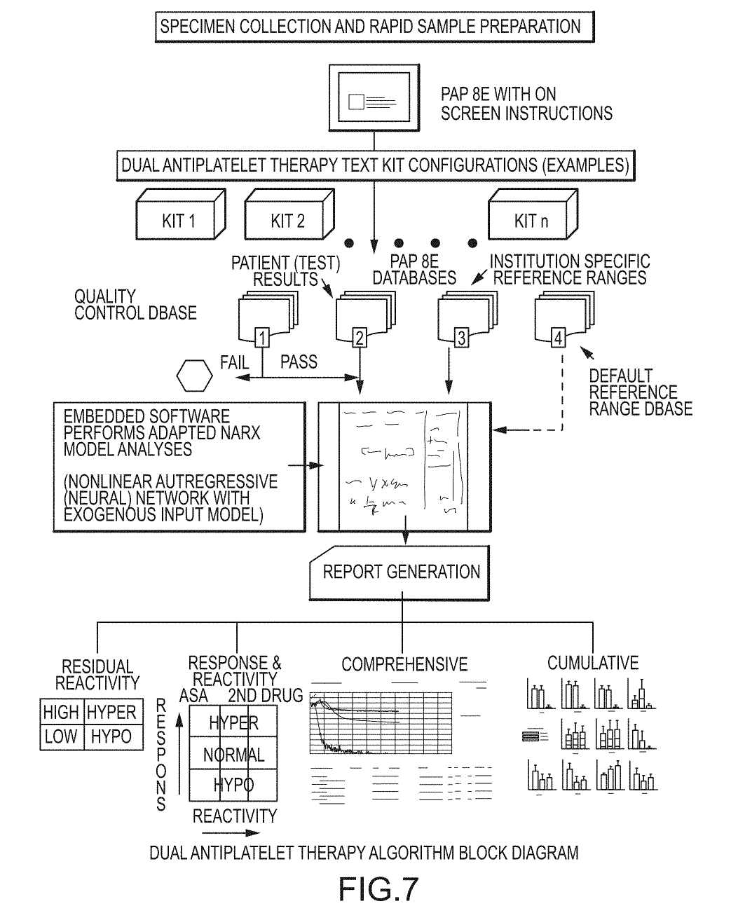

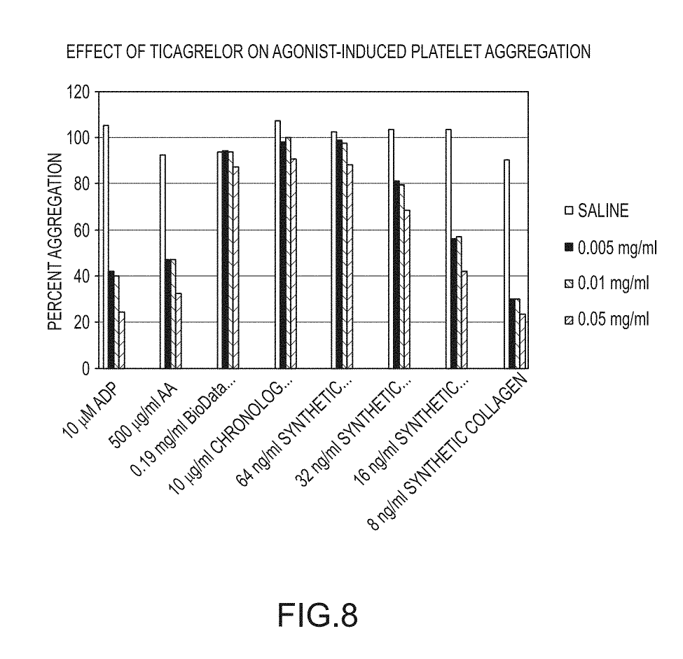

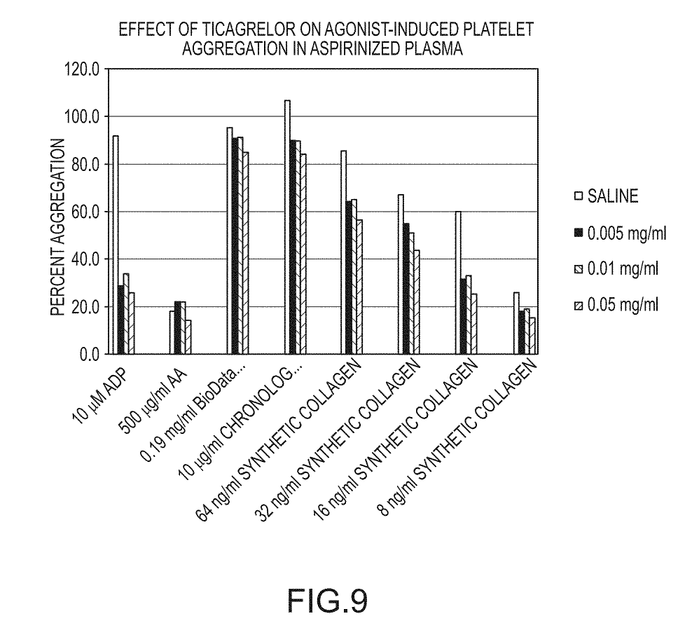

In non-aspirinized plasma, ADP-induced aggregation was strongly inhibited (FIG. 8). Arachidonic acid-induced aggregation was inhibited to a similar extent. Neither Bio/Data collagen, Chrono Log collagen nor 64 ng/mL synthetic collagen was markedly impacted by ticagrelor. The antiplatelet effects of Ticagrelor® could be identified when aggregation was induced by synthetic collagen at concentrations of 32 ng/mL and lower. The sensitivity for ticagrelor detection appeared to increase with decreasing synthetic collagen concentration. The addition of aspirin did not affect the ADP-induced aggregation response (FIG. 9). In contrast, an anti-platelet effect was no longer observed in arachidonic acid and 8 ng/mL synthetic collagen-induced aggregation assays and the effect in 16 and 32 ng/mL synthetic collagen-induced aggregation assays was reduced.

### Cilostazol

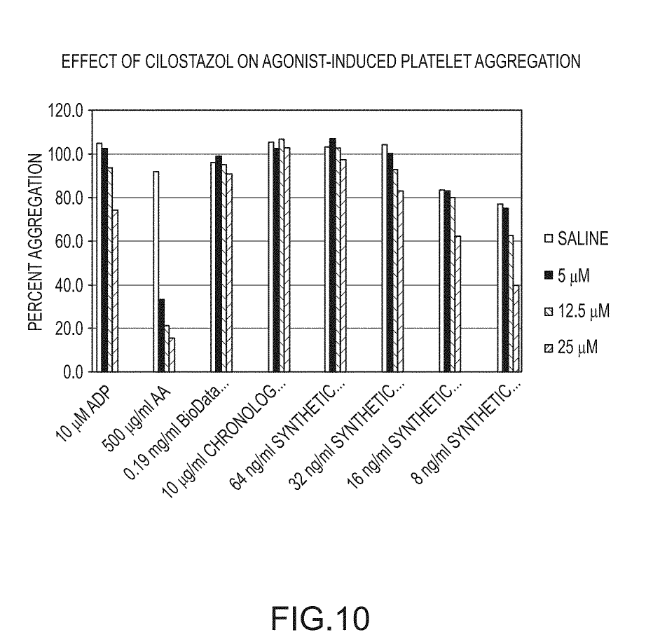

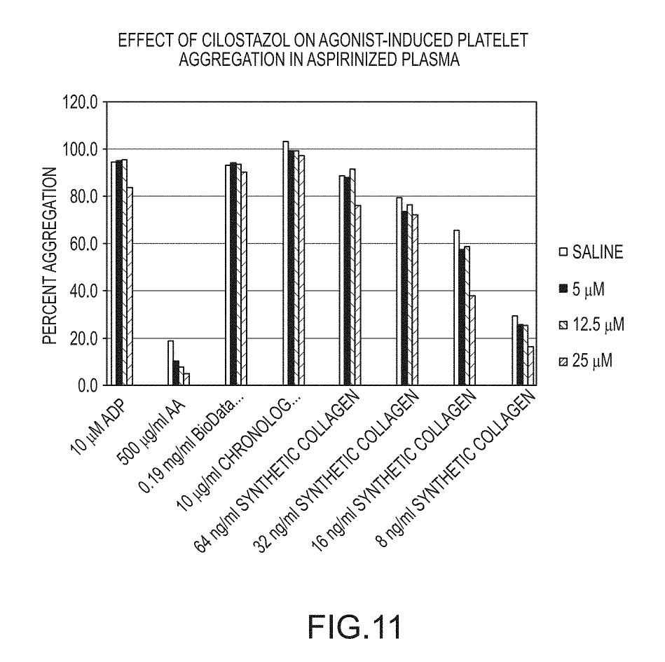

In non-aspirinized plasma, the most marked effect of cilostazol was on arachidonic acid-induced aggregation (FIG. 10), where aggregation levels of ˜20% were observed at concentrations ≥12.5 μM (vs. 95% in the absence of cilostazol). ADP-induced aggregation was minimally affected (˜30% inhibition at 25 μM). Cilostazol at concentrations up to 25 μM did not inhibit aggregation induced by Bio/Data collagen, Chrono Log collagen or the 64 ng/mL concentration of synthetic collagen. At lower concentrations of synthetic collagen, the anti-platelet effect of higher concentrations of cilostazol could be observed. Addition of aspirin to the PRP negated the ability to detect cilostazol (FIG. 11).

### Abciximab

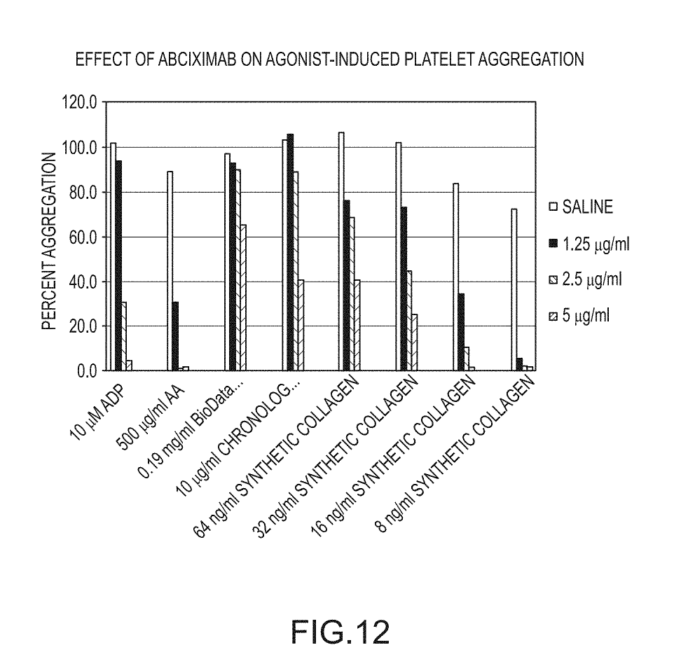

In non-aspirinized plasma, abciximab produced a concentration-dependent inhibition of agonist-induced aggregation over the concentration range tested (1.25-5 μg/mL) (FIG. 12). Arachidonic acid and synthetic collagen (8 and 16 ng/mL) were the most sensitive for detecting the presence of abciximab. Inhibition of Bio/Data and Chrono Log collagen-induced aggregation was only observed at the 5 μg/mL concentration.

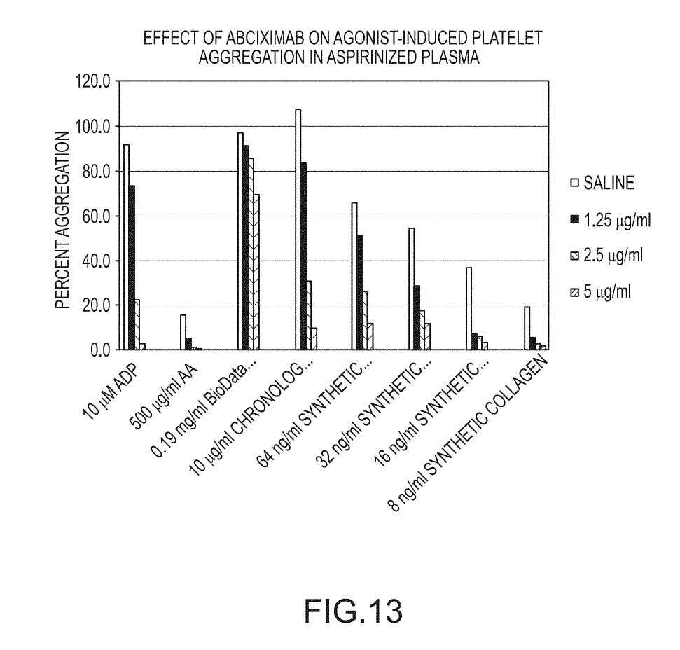

In aspirinized plasma, the response to arachidonic acid was nearly completely blunted (<20% aggregation) (FIG. 13). The addition of abciximab to aspirinized plasma resulted in complete inhibition of arachidonic acid-induced aggregation. Aggregation induced by 10 μM ADP and by 0.19 mg/mL Bio/Data collagen appeared to me minimally affected by the presence of aspirin. Although, aspirin did not inhibit 10 μg/mL Chrono Log collagen-induced aggregation, abciximab produced a stronger inhibition of Chrono Log collagen-induced aggregation in aspirinized PRP. At all concentrations tested, the synthetic collagen reagent produced lower levels of aggregation in aspirinized plasma than in non-aspirinized plasma.

### Discussion:

ADP, arachidonic acid and biological collagen are commonly used agonists to study platelet function. Ticagrelor inhibited aggregation induced by ADP and arachidonic acid, but had little effect on biological collagen-induced aggregation. Cilostazol strongly inhibited arachidonic acid-induced aggregation and produced a weaker, concentration-dependent inhibition of ADP-induced aggregation. Biological collagen-induced aggregation was unaffected by cilostazol. Abciximab inhibited aggregation induced by ADP, arachidonic acid and biological collagen, though ADP and arachidonic acid were more sensitive.

The synthetic collagen reagent was tested at concentrations ranging from 8 to 64 ng/mL. Aggregation induced by the 64 ng/mL concentration of the synthetic collagen was comparable to that of the Bio/Data and Chrono Log biological collagen reagents in that there was minimal effect of ticagrelor or cilostazol on the aggregation response. Aggregation induced by collagen or the 64 ng/mL synthetic collagen was inhibited by abciximab to a comparable degree. At lower concentrations of synthetic collagen, the antiplatelet effect of ticagrelor, cilostazol and abciximab were readily apparent.

At the concentrations tested, aggregation induced by Bio/Data collagen or Chrono Log collagen was not inhibited by aspirin. While the synthetic collagen reagent exhibited a higher sensitivity to aspirin in the absence of other antiplatelet drugs than either biological collagen, the presence of aspirin affected the ability of the synthetic collagen at these concentrations to detect the presence of ticagrelor or cilostazol.

### Conclusion:

Abciximab is readily detectable in the presence of aspirin using the synthetic collagen concentrations provided for this study. Ticagrelor can be detected, but the concentration-dependence isn't as well defined as it is with abciximab.

### Example 2: Testing for Platelet Aggregation Using LTAAs and Synthetic Collagen and Adding Anti-Platelet Medication to the PRP Sample

### Abciximab (ReoPro®)

Two healthy donors supplied the PRP samples. A panel of four different synthetic collagen concentrations were tested (12.5 ng/mL, 25 ng/mL, 50 ng/mL and 100 ng/mL Abciximab was added to the each of the four panels of different synthetic collagen concentrations. In one panel, saline was added as the control to each of the four synthetic collagen concentrations. In the second panel, 12.5 μg/mL (micrograms/mL) of abciximab was added to each of the four synthetic collagen concentrations. In a third panel, 25 μg/mL of abciximab was added to each of the four synthetic collagen concentrations. In a fourth panel, 50 μg/mL of abciximab was added to each of the four synthetic collagen concentrations. LTAAs were run for each and the PA, PS, SA, SS AUC, LP, DA, MA and FA was measured for each. The tests were run on each of the donors' samples.

### Ticagrelor (Brilinta®)

Two healthy donors supplied the PRP samples. A panel of four different synthetic collagen concentrations were tested (12.5 ng/mL (nanograms/mL), 25 ng/mL, 50 ng/mL and 100 ng/mL. Ticarelor was added to the each of the four panels of different synthetic collagen concentrations. In one panel, saline was added as the control to each of the four synthetic collagen concentrations. In the second panel, 0.05 mg/mL (milligrams/mL) of Ticagrelor was added to each of the four synthetic collagen concentrations. In a third panel, 0.1 mg/mL of Ticagrelor was added to each of the four synthetic collagen concentrations. In a fourth panel, 0.5 mg/mL of Ticagrelor was added to each of the four synthetic collagen concentrations. LTAAs were run for each and the PA, PS, SA, SS AUC, LP, DA, MA and FA was measured for each. The tests were run on each of the donors' samples.

The results showed that the synthetic collagen detected the drugs at the four different concentrations. The synthetic collagen and the two drugs caused the platelets to aggregate. The results also showed that the different dilutions of the two drugs worked with all four dilutions of synthetic collagen to cause the platelets to aggregate. The tests also revealed that certain combinations of synthetic collagen in combination with certain drug dilutions were more sensitive than others.

Abciximab and Ticagrelor are two different classes of drugs and yet the assays worked with both.

In another test, the samples could also be aspirinized to test for the effect of the dual therapy of the anti-platelet medication and the aspirin on a donor's platelets.

### Example 3: Use of Flow Cytometry to Test for Platelet Aggregation

A vial of Bio/Data calf skin collagen was reconstituted with 0.5 ml of water to make a 1.9 mg/ml solution. A vial of synthetic collagen was reconstituted with 1 ml of synthetic collagen diluent to make a 0.0005 mg/ml solution. Chrono Log collagen was diluted with saline to make a 100 μg/ml solution. A stock 2% paraformaldehyde solution was diluted with calcium-free Tyrode's buffer to make a 1% paraformaldehyde solution. A set of tubes containing 1 ml of 1% paraformaldehyde was prepared. A second set of tubes which contained 30 μl of collagen reagent and 30 μl of antiplatelet drug was prepared and set in a 37° C. heating block. Whole blood was drawn from healthy individuals into sodium citrate. 240 μl of citrated blood was added to the tubes at 15-20 second intervals and gently mixed. After a 3 minute incubation period, 50 μl of activated blood was transferred to the corresponding paraformaldehyde-containing tube. After a 30 minute incubation at 4° C., the samples were centrifuged at 1,600 rpm for 10 minutes and the supernatant was removed. The cell pellet was resuspended in 750 μl of Tyrode's buffer. 10 μl each of CD61FITC and CD62PE (BD Biosciences) was added to a set of clean tubes. 100 μl of resuspended cells was added to the antibody tubes. After a 30 minute incubation period in the dark at room temperature, 700 μl of Tyrode's buffer was added to each tube and the samples were analyzed on the flow cytometer (EPICS-XL, Beckman-Coulter). Platelet activation was assessed in terms of the percentage of platelets expressing P-selectin and the percentage of aggregated platelets.

1. A platelet aggregation assay comprising a light transmission aggregometry assay for determining an individual's anti-platelet medication sensitivity status, when the individual is on a dual therapy of aspirin and an anti-platelet medication that is not aspirin, the assay comprising the use of synthetic collagen at a final concentration in the light transmission aggregometry assay from about 40 ng/mL to about 500 ng/mL,
wherein the synthetic collagen comprises a polypeptide having a peptide fragment represented by the formula (I)
-(Pro-X-Gly)ₙ (I)
wherein X represents Hyp; and n represents an integer of from 20 to 250.

2. The method of claim 1 wherein platelet aggregation is analyzed with light transmission aggregometry assays and wherein the patient sample is platelet rich plasma.
